(19) United States
(12) Patent Application Publication
(10) Pub. No.: US 2023/0053994 A1
(43) Pub. Date: Feb. 23, 2023

(54) METHODS FOR MEASURING GUT PERMEABILITY AND GASTRIC EMPTYING RATE

(71) Applicant: IMPERIAL COLLEGE INNOVATIONS LIMITED, London (GB)

(72) Inventor: Alexander J. THOMPSON, London (GB)

(21) Appl. No.: 17/759,602

(22) PCT Filed: Jan. 29, 2021
(86) PCT No.: PCT/EP2021/052175

§ 371 Date: Jul. 27, 2022

(30) Foreign Application Priority Data
Jan. 31, 2020 (GB) 2001336.3

(51) Int. Cl. A61B 5/00

(52) U.S. Cl. CPC A61B 5/0071; A61B 5/4255; A61B 5/4238; A61B 5/6813

(57) ABSTRACT

A method for measuring gut permeability includes administering a solution including a fluorescent contrast agent to a subject; irradiating a location on the skin of the subject to cause a portion of the solution leaked into the bloodstream to fluoresce; obtaining fluorescence data of the intensity of the fluorescence as a function of time; normalising the fluorescence data to obtain normalised data of said intensity as a function of time; and analysing said normalised data to determine the gut permeability by calculating: (a) the first peak value of said intensity; (b) the integral of said intensity with respect to time; (c) the product of the first peak value of said intensity and the time at said peak value; (d) the time at which the first peak value of said intensity occurs; or (e) the first peak value of said intensity divided by the time at which said peak value occurs.

### CROSS-REFERENCE TO RELATED APPLICATIONS

The present application is the U.S. National Stage Application under 35 U.S.C. § 371 of International Application No. PCT/EP2021/052175, filed on Jan. 29, 2021, which claims the benefit of priority to United Kingdom Application No. GB2001336.3, filed on Jan. 31, 2020, the disclosures of which are hereby incorporated by reference in their entireties.

### FIELD

The present disclosure relates to a method for measuring gut permeability of a subject, and to a method for measuring gastric emptying rate of a subject.

### BACKGROUND

The function of the gastro-intestinal tract plays a pivotal role in countless health disorders including coeliac disease, inflammatory bowel disease (IBD), human immunodeficiency virus (HIV), fatty liver disease, sepsis, chronic liver disease, environmental enteric dysfunction (EED) and malnutrition. In particular, disruption of the gut barrier has important effects in the above conditions, leading to increased intestinal permeability (also referred to as “leakiness”) and even translocation of gut bacteria and pathogen-associated molecular peptides into systemic circulation causing inflammatory responses.

For example, increased gut permeability (also referred to as “leaky gut”) occurs when the intestinal barrier becomes compromised and involves the leakage of bacteria and other pathogens out of the intestine into lymph and blood vessels. This results in a cycle of infection and inflammation that can drive additional consequences and further exacerbate the issue of increased intestinal permeability. The condition is associated with many of the above diseases, and has a significant impact on quality of life and restricts physical and cognitive development of children with malnutrition.

Furthermore, the role and impact of the gut and of intestinal barrier function in many of the conditions highlighted above is poorly understood.

Similarly, altered gastric emptying rate (the rate at which food empties from the stomach into the intestine) is observed in numerous diseases including functional dyspepsia, gastroparesis and stomach cancer. In addition, gastric emptying rate is important in human nutrition, as it determines the rate at which nutrients can be absorbed.

Current techniques for assessing gut and gastro-intestinal function (particularly gut permeability and gastric emptying rate) can be cumbersome, expensive, highly invasive (for example, requiring endoscopic biopsy and histopathology or x-ray examinations), unreliable (for example, using urine and/or blood samples) and difficult to perform in infants.

Thus, there is a need for new technologies that can enhance the understanding of gastro-intestinal function in the pathogenesis of disease and that can serve to provide earlier diagnosis and improved monitoring of the many illnesses in which it plays a role. In particular, there is a need for improved means for assessing and quantifying gut permeability and for improved means for assessing and quantifying gastric emptying rate.

WO2015070256 discloses compositions and methods for assessing gut function. A composition for assessing gut function is disclosed, the composition comprising a fluorescent challenge molecule, wherein the fluorescent challenge molecule is not substantially absorbed by a healthy gut. Also disclosed is a composition for assessing gut function comprising at least two fluorescent challenge molecules, wherein one fluorescent challenge molecule is not substantially absorbed by a healthy gut, and the other is substantially absorbed by a healthy gut.

WO2010020673 discloses a sensor plaster for the transcutaneous measurement of the functioning of an organ, particularly the functioning of a kidney. The sensor plaster has at least one flexible support element with at least one adhesive surface which can adhere to a body surface. The sensor plaster also has at least one radiation source, particularly a light source, wherein the radiation source is set up to irradiate the body surface with at least one interrogation light. The sensor plaster also has at least one detector which is set up to detect at least one response light radiating from the direction of the body surface.

The present disclosure seeks to alleviate, at least to a certain degree, the problems and/or address at least to a certain extent, the difficulties associated with the prior art.

### SUMMARY

According to a first aspect of the disclosure, there is provided a method for measuring gut permeability of a subject, the method comprising:

orally administering a solution comprising a fluorescent contrast agent that is absorbable by a healthy gut to the subject;

using a light source to irradiate a location on the skin on a body part of the subject using light radiation, such that the light radiation causes at least a portion of the solution which has leaked out of the gut of the subject into the bloodstream of the subject to fluoresce;

using a transcutaneous sensing device to periodically detect the intensity of the fluorescence of the solution at said location to obtain fluorescence data of said intensity as a function of time;

normalising the fluorescence data to obtain normalised data of said intensity as a function of time; and

analysing said normalised data to determine the gut permeability of the subject by calculating one or more of:
the first peak value of said intensity;
the integral of said intensity with respect to time;
the product of the first peak value of said intensity and the time at said peak value;
the product of: the first peak value of said intensity, and the time at a point past the time of said first peak value;
the time at which the first peak value of said intensity occurs; or
the first peak value of said intensity divided by the time at which said peak value occurs.

Advantageously, such a method can provide for a minimally invasive, reliable and quantifiable way to assess the gut permeability of a subject.

In particular, using a fluorescent contrast agent that is absorbable by a healthy gut can advantageously provide that a baseline measurement can be obtained, such that even small changes in permeability can be detected, without the need for using multiple different fluorescent contrast agents having different molecular properties. As such, using a fluorescent contrast agent that is absorbable by a healthy gut can provide for diagnostic measurements to be obtained in all individuals, whether they have a healthy or unhealthy gut. Advantageously, this means that all disease states can be assessed, not just disease states beyond an arbitrary threshold above which the fluorescent contrast agent is first absorbed, as would be the case if a fluorescent contrast agent that was not absorbable by a healthy gut was used instead. Accordingly, such a method may advantageously be used to assess guts which are only slightly damaged, rather than just guts which are very damaged, and can provide a quantitative indication of how damaged a gut is, rather than just a binary indication of whether it is damaged or not damaged.

Optionally, the fluorescence data is normalised with respect to acquisition time and the intensity of the backscattered light radiation, i.e. of the backscattered excitation light.

Optionally, the normalised data is analysed to determine the gut permeability of the subject by calculating the integral of said intensity with respect to time up until the time at which the first peak value of said intensity occurs.

Furthermore, normalising the fluorescence data with respect to the intensity of the backscattered excitation light (i.e. the intensity of the signal from the light source (such as a laser, light emitting diode or other light source) that is used to excite the fluorescence) and analysing said normalised data to determine the gut permeability of the subject by calculating one or more of: the first peak value of said intensity; the integral of said intensity with respect to time (up until a chosen time point, for example: the time of the first peak, or the sum of the time of the first peak plus a chosen percentage of said time of the first peak, for example 5, 10 or 20% thereof, i.e. at a point past the time of the first peak); the product of the first peak value of said intensity and the time at said peak value; the product of the first peak value of said intensity and the time at a point past the time of said first peak value; the time at which the first peak value of said intensity occurs; or the first peak value of said intensity divided by the time at which said peak value occurs, can advantageously provide that a quantitative assessment of the gut permeability can be obtained, by the use of only a single fluorescent contrast agent (i.e. without the need for the solution to comprise two of more different fluorescent contrast agents, such as two different fluorescent contrast agents having different molecular weights). Thus, continuous measurements of gut permeability can be obtained by using a single fluorescent contrast agent.

The first peak value of said intensity can be used to determine the gut permeability of the subject, because this peak value can provide a readout of the peak concentration of the fluorescent contrast agent in the bloodstream of the subject. Said peak value can represent the point in time at which there is a maximum concentration of the fluorescent contrast agent in the bloodstream of the subject and may typically occur before significant elimination of the solution from the body has occurred (for example, via the kidneys or liver).

Accordingly, overall, such a method can provide for the quantification of gut permeability based on the normalised fluorescence intensity data, and also provide an indication of other aspects of gastro-intestinal (GI) function, without the need for collecting urine, blood or other samples. This can allow for quantification of the leakage of dyes that pass even the healthy gut barrier and means that it can be possible to provide meaningful clinical data in a wide range of patients (i.e. subjects) and not just those with extremely permeable/damaged guts. In addition, the quantifiers/calculations in such a method can provide measurements that vary in a continuous manner with respect to gut permeability, rather than simple binary markers (as in the prior art) that can provide only coarse clinical outcomes of whether a gut is “permeable” or “not permeable”. This can permit monitoring of patients' responses to therapies and other interventions (such as nutritional interventions) and can also allow for comparison of values measured in different individuals (i.e. for screening/diagnostic applications). This can be possible due to the normalisation of the fluorescence data (with respect to the backscattered excitation signal) and due to the periodic collection of data.

Furthermore, such a method demonstrates the potential for non-invasive sensing of gut permeability, and can provide for the development of miniaturised, low-cost, wearable sensors suitable for field-deployable use.

Optionally, the normalised data is analysed to determine the gut permeability of the subject by calculating the integral of said intensity with respect to time up until a chosen time point after the first peak value of said intensity.

For example, said chosen time point may be equal to the sum of the value of time at which the first peak value of said intensity occurs, plus a chosen percentage of said value of time. Said chosen percentage may be between approximately 5% to 25%, for example, said chosen percentage may be approximately 5%, 10% or 20%. For example, if the first peak value of said intensity is observed at a time value of 60 minutes, then the data could be integrated up until a time of 63 minutes, 66 minutes or 72 minutes, for chosen percentage values of time adjustment of 5%, 10% and 20% respectively. Advantageously, integrating the data up until a time point slightly past the time of the first peak value of said intensity can provide that the resulting analysis accounts for elimination of the contrast agent from the blood stream of the subject. The later said first peak occurs (in time), the more elimination of the contrast agent from the body will have occurred, therefore the more important and advantageous such a correction becomes if said first peak occurs later. Advantageously, integrating up to slightly past the peak corrects for this.

Optionally, the solution contains only a single fluorescent contrast agent that is absorbable by a healthy gut. That is, the solution may contain said fluorescent contrast agent that is absorbable by a healthy gut, and no other fluorescent contrast agents.

Optionally, to calculate the first peak value of said intensity, the equation GP₁=I(tₚₑₐₖ) may be used, wherein GP₁ represents a quantifier of gut permeability and I(tₚₑₐₖ) represents the value of the normalised data of said intensity as a function of time at the time (tₚₑₐₖ) of the initial peak in said intensity data.

The integral of said intensity with respect to time (i.e. the area under the curve of said intensity plotted against time) can be used to determine the gut permeability of the subject, accounting for the effects of gastric emptying rate. Optionally, the integral can be calculated up to the first peak of said intensity. Optionally, the integral can be calculated up to a chosen time point past the first peak of said intensity. This can provide a quantitative measurement of the total amount of the fluorescent contrast agent that has leaked from the gut into the bloodstream of the subject up to the time of said first peak (or up to an alternative chosen time point, for example a chosen time point past the time of said first peak). By identifying the time of peak concentration from the data, it can be possible to make an accurate quantification of the total recovery of the fluorescent contrast agent in the bloodstream of the subject.

Optionally, to calculate the integral of said intensity with respect to time, the equation GP₂=Σₜ₌₀^{t}^{ₚₑₐₖ}I(t), may be used, wherein GP₂ represents a quantifier of gut permeability, I(t) represents the normalised data of said intensity as a function of time, and tₚₑₐₖ represents the time of the initial peak in the fluorescence data. Alternatively, the equation for GP₂ may be modified as Gp₂=Σₜ₌₀^{t}^{ₚₑₐₖ₊}I(t), wherein tₚₑₐₖ₊ represents a chosen time point after the first peak in the fluorescence data. For example, said chosen time point may be equal to the sum of the value of time at which the initial peak in the fluorescence data occurs, plus a chosen percentage of said value of time. Said chosen percentage may be between approximately 5% to 25%, for example, said chosen percentage may be approximately 5%, 10% or 20%.

The product of the first peak value of said intensity and the time at said peak value can be used to determine the gut permeability of the subject, and can advantageously represent a simplified analysis approach to calculating the integral of said intensity with respect to time.

Alternatively, the product of the first peak value of said intensity and the time at a point past the time of said peak value can be used to determine the gut permeability of the subject. This can also advantageously represent a simplified analysis approach to calculating the integral of said intensity with respect to time. The point past the time of said peak value may be defined as the sum of the time of the first peak plus a chosen percentage of said time of the first peak, for example 5, 10 or 20% thereof (i.e. the time is defined as a point past/after the time of the first peak). For example, if the first peak value of said intensity is observed at a time value of 60 minutes, then said point could be chosen to be a time value of 63 minutes, 66 minutes or 72 minutes, for chosen percentage values of time adjustment of 5%, 10% and 20% respectively. Advantageously, taking the product of: the intensity at the first peak value, and the time at a point slightly past the time of the first peak value of said intensity, can provide that the resulting analysis accounts for elimination of the contrast agent from the blood stream of the subject. The later said first peak occurs (in time), the more elimination of the contrast agent from the body will have occurred, therefore the more important and advantageous such a correction becomes if said first peak occurs later. Advantageously, taking the product of the first peak value of intensity and the time at a point slightly past the peak corrects for this.

Optionally, to calculate the product of the first peak value of said intensity and the time at said peak value, the equation GP₃=I(tₚₑₐₖ)×tₚₑₐₖ may be used; wherein GP₃ represents a quantifier of gut permeability, I(tₚₑₐₖ represents the value of the normalised data of said intensity as a function of time at the time (tₚₑₐₖ) of the initial peak in said intensity data; and tₚₑₐₖ represents the time of the initial peak in the fluorescence data. Alternatively, to calculate the product of: the first peak value of said intensity, and the time at a point past the time of said first peak value, the above equation for GP₃ may be modified as GP₃=I(tₚₑₐₖ)×tₚₑₐₖ₊; wherein tₚₑₐₖ₊ represents a chosen time point after the initial peak in +-peak the fluorescence data, for example a time 5, 10 or 20% past the time of said initial peak.

The time at which the first peak value of said intensity occurs, or the first peak value of said intensity divided by the time at which said peak value occurs can be used to determine the gut permeability of the subject, with lower values of time at which said peak value occurs corresponding to higher permeabilities.

Optionally, to calculate the time at which the first peak value of said intensity occurs, the equation GP₄=tₚₑₐₖ may be used, wherein GP₄ represents a quantifier of gut permeability and represents the time of the initial peak in the fluorescence data.

Optionally, to calculate the first peak value of said intensity divided by the time at which said peak value occurs, the equation

$${GP}_{5} = \frac{I ( {t}_{peak} )}{{t}_{peak}}$$

may be used, wherein GP₅ represents a quantifier of gut permeability, I(tₚₑₐₖ) represents the value of the normalised data of said intensity as a function of time at the time (tₚₑₐₖ) of the initial peak in said intensity data, and tₚₑₐₖ represents the time of the initial peak in the fluorescence data.

Advantageously, GP₂ and GP₃ can provide a degree of correction to account for gastric emptying rates and thus may offer a more quantitative assessment of permeability.

Optionally, the step of using a transcutaneous sensing device to periodically detect the intensity of the fluorescence of the solution at said location to obtain fluorescence data of said intensity as a function of time comprises using said transcutaneous sensing device to measure the intensity of the fluorescence of the solution and also to measure the backscattered intensity of said light radiation used to irradiate said location on the skin on said body part.

Optionally, the step of using a transcutaneous sensing device to periodically detect the intensity of the fluorescence of the solution at said location to obtain fluorescence data of said intensity as a function of time comprises measuring the backscattered light simultaneously with detecting said fluorescence.

Optionally, the measurement of said backscattered light is measured at the same location to that at which the intensity of the fluorescence of the solution is periodically detected, i.e. at said location on the skin on said body part.

Optionally, the measurement of said backscattered light is measured at a different location to that at which the intensity of the fluorescence of the solution is periodically detected, for example at a different location which is in close proximity to but spaced apart from said location on the skin on said body part.

Optionally, said different location is a location on the skin of the subject. Optionally, this may be provided by means of a fibre-optic probe with a filter wheel at the proximal end to provide switching between fluorescence and laser (excitation) measurements, which can advantageously provide an identical measurement location but different measurement times. Optionally, this may alternatively be provided by means of a wearable sensor with one excitation source (e.g. an LED) and two very closely positioned detectors (one to detect the fluorescence signal, the other to detect the excitation signal), which can advantageously provide near identical measurement locations and identical measurement times.

Advantageously, this can provide that the data can be correctly normalised to account for any fluctuations between patients and/or between repeat measurements in the same patient etc.

Advantageously, performing the normalisation with respect to excitation power by recording the intensity of the backscattered excitation signal at a location on the skin rather than at the light source itself can allow for correction for variations due to skin tone, skin thickness, and/or movement of the light source etc. to be made.

Optionally, the solution comprises water or juice.

Advantageously, delivering the fluorescent contrast agent to the subject as a solution in water or juice can ensure that no other constituents of the drink affect the gut permeability. In younger patents who may find the solution too bitter in taste, the solution can be dissolved in orange juice.

Optionally, the contrast agent comprises a dye, for example fluorescein, methylene blue or fluorescein isothiocyanate conjugated dextran, or salts thereof, or combinations thereof.

Advantageously, fluorescein is taken up rapidly in the small intestine, after it empties out of the stomach, and is a clinically approved dye. Thus, the rate of uptake of fluorescein into the bloodstream (and the other permeability quantifiers described above) can be used to assess gut permeability.

Advantageously, methylene blue is taken up rapidly in the small intestine, after it empties out of the stomach, and is a clinically approved dye. Thus, the rate of uptake of methylene blue into the bloodstream (and the other permeability quantifiers described above) can be used to assess gut permeability.

Optionally, the solution comprises a dosage of approximately 100 to 500 mg of the contrast agent.

Advantageously, such a dosage of the contrast agent can ensure good signal-to-noise ratios for the fluorescence data, while remaining within clinically acceptable levels.

Optionally, the solution comprises a dosage of at least 25 mg of fluorescein as the fluorescent contrast agent.

Optionally, the solution comprises a dosage of approximately 100 mg of the contrast agent.

Optionally, the solution comprises a dosage of 100 mg of fluorescein or methylene blue as the fluorescent contrast agent.

Optionally, the solution comprises a dosage of approximately 100 to 500 mg of fluorescein as the fluorescent contrast agent, in approximately 100 ml of water or juice.

Optionally, the solution comprises a dosage of between 25 mg to 500 mg of the contrast agent.

Optionally, the solution comprises a dosage of up to 500 mg of fluorescein as the contrast agent.

Optionally, the solution comprises a dosage of up to 100 mg of methylene blue as the contrast agent.

The dosage of the fluorescent contrast agent in the solution may be chosen to find the optimum balance between minimising the dose and maximising the signal-to-noise ratio.

Optionally, the transcutaneous sensing device has an acquisition time and the light source has an excitation power, and the fluorescence data is normalised based on said acquisition time and said excitation power.

Advantageously, normalising the fluorescence intensity value for each time point according to the acquisition time and excitation power used in each case can allow all the time points to be compared against one another. Advantageously, this can allow measurements from different days or times or in different subjects to be quantitatively compared. Furthermore, this can provide that the fluorescence values can be used to provide meaningful quantifications of variations in permeability (for example, across different patients or over time in one patient), rather than simply providing a binary assessment as to whether or not a contrast agent has passed the gut barrier.

Optionally, the transcutaneous sensing device has an acquisition time of between 100 ms to 15 s.

Optionally, the transcutaneous sensing device has an acquisition time of at least 100 ms.

Optionally, the transcutaneous sensing device is configured such that the acquisition time for each measurement can be determined in an automated manner by said transcutaneous sensing device itself. That is, optionally, the transcutaneous sensing device may be configured to determine the optimal acquisition time for each time point based on the current level of the fluorescence and/or backscattered excitation signal. Optionally, the fluorescence data of said intensity as a function of time may then be normalised according to the acquisition times used, for collection of both the fluorescence data and the backscattered excitation data.

Advantageously, this can ensure that good signal levels are acquired at all time points, even when the fluorescence levels are low (for example at the beginning or end of the measurement/detection process). This can also ensure that there are no problems related to detector saturation when the fluorescence signal is at its highest. Advantageously, this can allow for measurements to be made reliably across patients with different skin tones, without the need for requiring higher doses of the fluorescent contrast agent in subjects with darker skin. Instead of requiring higher doses or higher excitation powers which may not be clinically acceptable, the transcutaneous sensing device can automatically acquire data with longer integration times to allow for suitable signal levels to be obtained.

Optionally, the transcutaneous sensing device is configured to detect the intensity of said fluorescence using fluorescence spectroscopy.

Optionally, the transcutaneous sensing device begins taking periodic measurements before the solution is administered to the subject, such that the periodic detection of the intensity of the fluorescence of the solution begins before the solution is administered to the subject, to obtain a background signal to be used in the step of normalising the fluorescence data.

Optionally, the background signal is subtracted from the fluorescence data of said intensity as a function of time for each time point (i.e. for each periodic measurement detected/taken).

Optionally, the fluorescence data is normalised based on the backscattered intensity of said light radiation.

Advantageously, such normalisation of the fluorescence data can provide that said fluorescence data is comparable across different measurements and across different subjects by using just a single fluorescent contrast agent.

Optionally, the light source comprises a light emitting diode or a laser.

Optionally, the light source is configured to be worn on and/or around said body part of the subject.

Optionally, the transcutaneous sensing device comprises one or more photodiodes, phototransistors and/or fibre-optic probes and is configured to be worn on and/or around said body part of the subject.

Optionally, in the step of using the transcutaneous sensing device to periodically detect said intensity of the fluorescence, measurements are recorded by said transcutaneous sensing device at least once every five minutes.

Optionally, in the step of using the transcutaneous sensing device to periodically detect said intensity of the fluorescence, measurements are recorded by said transcutaneous sensing device at least one every two minutes.

Optionally, in the step of using the transcutaneous sensing device to periodically detect said intensity of the fluorescence, measurements are recorded by said transcutaneous sensing device at least once per minute.

Advantageously, the frequency of said measurements may be chosen to ensure that the data collected has sufficient temporal resolution. Recording measurements at least once every five minutes can ensure that this is the case. This can help to accurately identify the first peak value of said intensity, which represents the peak concentration of the fluorescent contrast agent in the bloodstream of the subject.

Furthermore, taking measurements at a sufficiently high frequency, for example once every five minutes, once every two minutes, or once every minute, can provide that the data obtained can indicate specifically the locations in the gut at which leakage is occurring, compared with less frequent/regular periodic measurements.

Optionally, said body part is a finger, wrist, arm or earlobe.

Advantageously, taking the measurements using said transcutaneous sensing device at a location on the skin of a finger, wrist, arm or earlobe can provide that clear fluorescence of the fluorescent contrast agent is observed, even when short acquisition times are used.

Optionally, said body part is a fingertip.

Advantageously, taking the measurements using said transcutaneous sensing device at a location on the fingertip can provide that clear fluorescence of the fluorescent contrast agent is observed, even when short acquisition times are used, and can provide that the intensity of the fluorescence data is higher, due to the proximity of blood vessels to the surface of the skin on a fingertip. Taking measurements on the fingertip can also minimise the effect of dye leakage from blood vessels.

According to a second aspect of the disclosure, there is provided a method for measuring gastric emptying rate of a subject, the method comprising:

orally administering a test meal comprising a fluorescent contrast agent that is absorbable by a healthy gut to the subject;

using a light source to irradiate a location on the skin on a body part of the subject using light radiation, such that the radiation causes at least a portion of the fluorescent contrast agent in the test meal which has been emptied from the stomach of the subject and has entered the bloodstream of the subject to fluoresce;

using a transcutaneous sensing device to periodically detect the intensity of the fluorescence of the fluorescent contrast agent in the test meal at said location to obtain data of said intensity as a function of time;

normalising the fluorescence data to obtain normalised data of said intensity as a function of time; and

analysing said normalised data to calculate the percentage of the test meal which is remaining in the stomach of the subject as a function of time, based on the intensity as a function of time divided by a peak value of the intensity.

Advantageously, such a method can provide for a minimally invasive, reliable and quantifiable way to assess the gastric emptying rate of a subject.

In particular, using a fluorescent contrast agent that is absorbable by a healthy gut can advantageously provide that measurements can be obtained in all participants (i.e. in subjects with normal or increased intestinal permeability). This can allow that even small changes in gastric emptying rate can be detected and that gastric emptying rate can be measured in all participants regardless of their gastro-intestinal health status. It can also allow for only a single fluorescent contrast agent to be used for all subjects, without the need for using multiple different fluorescent contrast agents having different molecular properties. As such, using a fluorescent contrast agent that is absorbable by a healthy gut can provide for diagnostic measurements to be obtained in all individuals, whether they have a healthy or unhealthy gut. Advantageously, this means that all disease states can be assessed, not just disease states beyond an arbitrary threshold above which the fluorescent contrast agent is first absorbed, as would be the case if a fluorescent contrast agent that was not absorbable by a healthy gut was used instead. Accordingly, such a method may advantageously be used to assess guts which are only slightly damaged, rather than just guts which are very damaged, and can provide a quantitative indication of how damaged a gut is, rather than just a binary indication of whether it is damaged or not damaged. In particular, by using a fluorescent contrast agent that is absorbable by a healthy gut, rather than using a fluorescent contrast agent that is only absorbable by a damaged gut, such a method can advantageously provide that gastric emptying rate can be measured regardless of whether the subject has any other intestinal health issues.

Furthermore, normalising the fluorescence data and analysing said normalised data to calculate the percentage of the test meal which is remaining in the stomach of the subject as a function of time, can advantageously provide that a quantitative assessment of gastric emptying rate can be obtained, by the use of only a single fluorescent contrast agent (i.e. without the need for the test meal to comprise two of more different fluorescent contrast agents, such as two different fluorescent contrast agents having different molecular weights, and without the need to use different contrast agents in subjects with different states of intestinal health). Thus, continuous measurements of gastric emptying rate can be obtained by using a single fluorescent contrast agent.

Accordingly, overall, such a method can provide for the quantification of gastric emptying rate based on the normalised fluorescence intensity data, and also provide an indication of other aspects of gastro-intestinal (GI) function, without the need for collecting urine, blood or other samples. This can allow for quantification of the leakage of dyes that pass even the healthy gut barrier and means that it can be possible to provide meaningful clinical data in a wide range of patients (i.e. subjects) and not just those with extremely permeable/damaged guts. In addition, the quantifiers/calculations in such a method can provide measurements that vary in a continuous manner with respect to gastric emptying rate. This can permit monitoring of patients' responses to therapies and other inventions (such as nutritional interventions) and can also allow for comparison of values measured in different individuals (i.e. for screening/diagnostic applications). This can be possible due to the normalisation of the fluorescence data and due to the periodic collection of data.

Furthermore, such a method demonstrates the potential for non-invasive sensing of gastric emptying rate, and can provide for the development of miniaturised, low-cost, wearable sensors suitable for field-deployable use.

Optionally, said peak value of the intensity is the value of the intensity at a first peak or at a second peak in the fluorescence data, or is a maximum value of the intensity in the fluorescence data. This may be chosen based on the particular method of calculation used.

Optionally, said peak value of the intensity is the value of the intensity at a first peak in the fluorescence data.

Optionally, the test meal contains only a single fluorescent contrast agent that is absorbable by a healthy gut. That is, the test meal may contain said fluorescent contrast agent that is absorbable by a healthy gut, and no other fluorescent contrast agents.

Optionally, the step of using a transcutaneous sensing device to periodically detect the intensity of the fluorescence of the test meal at said location to obtain fluorescence data of said intensity as a function of time comprises using said transcutaneous sensing device to measure the intensity of the fluorescence of the test meal and also to measure intensity of the backscattered intensity of said light radiation.

Optionally, the step of using a transcutaneous sensing device to periodically detect the intensity of the fluorescence of the test meal at said location to obtain fluorescence data of said intensity as a function of time comprises using said transcutaneous sensing device to measure the intensity of the fluorescence of the test meal and also to measure the backscattered intensity of said light radiation used to irradiate said location on the skin on said body part.

Optionally, the step of using a transcutaneous sensing device to periodically detect the intensity of the fluorescence of the test meal at said location to obtain fluorescence data of said intensity as a function of time comprises measuring the backscattered light simultaneously with detecting said fluorescence.

Optionally, the measurement of said backscattered light is measured at the same location to that at which the intensity of the fluorescence of the test meal is periodically detected, i.e. at said location on the skin on said body part.

Optionally, the measurement of said backscattered light is measured at a different location to that at which the intensity of the fluorescence of the test meal is periodically detected, for example at a different location which is in close proximity to but spaced apart from said location on the skin on said body part.

Optionally, said different location is a location on the skin of the subject.

Advantageously, performing the normalisation with respect to excitation power by recording the intensity of the backscattered excitation signal at a location on the skin rather than at the light source itself can allow for correction for variations due to skin tone, skin thickness, movement of the light source etc. to be made.

Optionally, the fluorescent contrast agent is dispersed within the test meal.

Optionally, the test meal comprises a solution.

Optionally, the test meal comprises a liquid test meal or a solid test meal.

Optionally, the test meal comprises a milkshake or scrambled eggs on toast.

Optionally, the contrast agent comprises a dye, for example fluorescein, methylene blue or fluorescein isothiocyanate conjugated dextran, or salts thereof, or combinations thereof.

Advantageously, fluorescein is taken up rapidly in the small intestine, after it empties out of the stomach, and is a clinically approved dye. Thus, the rate of uptake of fluorescein into the bloodstream can be used to assess gastric emptying rate.

Advantageously, methylene blue is taken up rapidly in the small intestine, after it empties out of the stomach, and is a clinically approved dye. Thus, the rate of uptake of methylene blue into the bloodstream can be used to assess gastric emptying rate.

Optionally, the test meal comprises a dosage of approximately 100 to 500 mg of the contrast agent.

Advantageously, such a dosage of the contrast agent can ensure good signal-to-noise ratios for the fluorescence data, while remaining within clinically acceptable levels.

Optionally, the test meal comprises a dosage of at least 25 mg of fluorescein as the fluorescent contrast agent.

Optionally, the test meal comprises a dosage of approximately 100 mg of the contrast agent.

Optionally, the test meal comprises a dosage of 100 mg of fluorescein or methylene blue as the fluorescent contrast agent.

Optionally, the test meal comprises a dosage of approximately 100 to 500 mg of fluorescein as the fluorescent contrast agent, in approximately 100 ml of water or juice.

Optionally, the test meal comprises a dosage of between 25 mg to 500 mg of the contrast agent.

Optionally, the test meal comprises a dosage of up to 500 mg of fluorescein as the contrast agent.

Optionally, the test meal comprises a dosage of up to 100 mg of methylene blue as the contrast agent.

The dosage of the fluorescent contrast agent in the test meal may be chosen to find the optimum balance between minimising the dose and maximising the signal-to-noise ratio.

Optionally, the transcutaneous sensing device has an acquisition time and the light source has an excitation power, and the fluorescence data is normalised based on said acquisition time and said excitation power.

Advantageously, normalising the fluorescence intensity value for each time point according to the acquisition time and excitation power used in each case can allow all the time points to be compared against one another. Advantageously, this can allow quantitative measurements of gastric emptying rate as well as quantitative comparison of measurements made on different days or times or in different subjects. Furthermore, this can provide that the fluorescence values can be used to provide meaningful quantifications of variations in gastric emptying rate (for example, across different patients or over time in one patient), rather than simply providing a binary assessment as to whether or not a contrast agent is remaining in the stomach.

Optionally, the transcutaneous sensing device has an acquisition time of between 100 ms to 15 s.

Optionally, the transcutaneous sensing device has an acquisition time of at least 100 ms.

Optionally, the transcutaneous sensing device is configured such that the acquisition time for each measurement can be determined in an automated manner by said transcutaneous sensing device itself. That is, optionally, the transcutaneous sensing device may be configured to determine the optimal acquisition time for each time point based on the current level of the fluorescence and/or backscattered excitation signal. Optionally, the fluorescence data of said intensity as a function of time may then be normalised according to the acquisition times used, for collection of both the fluorescence data and the backscattered excitation data.

Advantageously, this can ensure that good signal levels are acquired at all time points, even when the fluorescence levels are low (for example at the beginning or end of the measurement/detection process). This can also ensure that there are no problems related to detector saturation when the fluorescence signal is at its highest. Advantageously, this can allow for measurements to be made reliably across patients with different skin tones, without the need for requiring higher doses of the fluorescent contrast agent in subjects with darker skin. Instead of requiring higher doses or higher excitation powers which may not be clinically acceptable, the transcutaneous sensing device can automatically acquire data with longer integration times to allow for suitable signal levels to be obtained.

Optionally, the transcutaneous sensing device is configured to detect the intensity of said fluorescence using fluorescence spectroscopy.

Optionally, the transcutaneous sensing device begins taking periodic measurements before the test meal is administered to the subject, such that the periodic detection of the intensity of the fluorescence of the fluorescent contrast agent in the test meal begins before the test meal is administered to the subject, to obtain a background signal to be used in the step of normalising the fluorescence data.

Optionally, the background signal is subtracted from the fluorescence data of said intensity as a function of time for each time point (i.e. for each periodic measurement detected/taken).

Optionally, the fluorescence data is normalised based on the backscattered intensity of said light radiation used to excite the fluorescence.

Advantageously, such normalisation of the fluorescence data can provide that said fluorescence data is comparable across different measurements and across different subjects by using just a single fluorescent contrast agent.

Optionally, the light source comprises a light emitting diode or a laser.

Optionally, the light source is configured to be worn on and/or around said body part of the subject.

Optionally, the transcutaneous sensing device comprises one or more photodiodes, phototransistors and/or fibre-optic probes and is configured to be worn on and/or around said body part of the subject.

Optionally, in the step of using the transcutaneous sensing device to periodically detect said intensity of the fluorescence, measurements are recorded by said transcutaneous sensing device at least once every five minutes.

Optionally, in the step of using the transcutaneous sensing device to periodically detect said intensity of the fluorescence, measurements are recorded by said transcutaneous sensing device at least once every two minutes.

Optionally, in the step of using the transcutaneous sensing device to periodically detect said intensity of the fluorescence, measurements are recorded by said transcutaneous sensing device at least once per minute.

Advantageously, the frequency of said measurements may be chosen to ensure that the data collected has sufficient temporal resolution. Recording measurements at least once every five minutes can ensure that this is the case. This can help to accurately identify the first peak value of said intensity, which represents the peak concentration of the fluorescent contrast agent in the bloodstream of the subject.

Furthermore, taking measurements at a sufficiently high frequency, for example once every five minutes or once every minute, can provide that the data obtained can indicate specifically the locations in the gut at which leakage is occurring, compared with less frequent/regular periodic measurements.

Optionally, in the step of using the transcutaneous sensing device to periodically detect said intensity of the fluorescence, measurements are recorded by said transcutaneous sensing device over a period of at least thirty minutes.

Optionally, in the step of using the transcutaneous sensing device to periodically detect said intensity of the fluorescence, measurements are recorded by said transcutaneous sensing device over a period of at least sixty minutes.

Optionally, in the step of using the transcutaneous sensing device to periodically detect said intensity of the fluorescence, measurements are recorded by said transcutaneous sensing device over a period of at least four hours.

Optionally, said body part is a finger, wrist, arm or earlobe.

Advantageously, taking the measurements using said transcutaneous sensing device at a location on the skin of a finger, wrist, arm or earlobe can provide that clear fluorescence of the fluorescent contrast agent is observed, even when short acquisition times are used.

Optionally, said body part is a fingertip.

Advantageously, taking the measurements using said transcutaneous sensing device at a location on the fingertip can provide that clear fluorescence of the fluorescent contrast agent is observed, even when short acquisition times are used, and can provide that the intensity of the fluorescence data is higher, due to the proximity of blood vessels to the surface of the skin on a fingertip. Taking measurements on the fingertip can also minimise the effect of dye leakage from blood vessels.

Optionally, the step of analysing said normalised data comprises fitting the function:

$$I ( t ) = ( 1 - Ct ) ( \frac{{B}_{max}}{1 + exp ( - {k}_{B} ( t - {t}_{B \frac{1}{2}} ) )} + \frac{{L}_{max}}{1 + exp ( - {k}_{L} ( t - {t}_{L \frac{1}{2}} ) )} )$$

onto the normalised data using a numerical fitting procedure such as least squares fitting;

wherein:

t represents time;

I(t) represents the normalised data of said fluorescence intensity as a function of time;

Bₘₐₓ represents the maximum value of the intensity contribution from the fluorescent contrast agent in the bloodstream of the subject as a function of time;

Lₘₐₓ represents the maximum value of the intensity contribution from the fluorescent contrast agent that has leaked into the epithelium of the skin of the subject as a function of time;

t_{B1/2} represents the time at which the intensity contribution from the fluorescent contrast agent in the bloodstream of the subject as a function of time reaches half of its maximum value;

t_{L1/2} represents the time at which the intensity contribution from the fluorescent contrast agent that has leaked into the epithelium of the skin of the subject as a function of time reaches half of its maximum value;

C represents the rate at which dye is eliminated from the body of the subject;

k_{B} is a constant and represents the logistic growth rate of the intensity contribution from the fluorescent contrast agent in the bloodstream of the subject as a function of time; and

k_{L} is a constant and represents the logistic growth rate of the intensity contribution from the fluorescent contrast agent that has leaked into the epithelium of the skin of the subject as a function of time.

Optionally, the step of analysing said normalised data further comprises calculating the amount of dye that has emptied from the stomach of the subject as a function of time, S(t), wherein S(t)=B(t)+B(t)Ct, wherein B(t) represents the intensity contribution from the fluorescent contrast agent in the bloodstream of the subject as a function of time.

Optionally, the step of analysing said normalised data further comprises calculating the percentage of the test meal which is remaining in the stomach of the subject as a function of time, R_{pc}, wherein:

$${R}_{pc} = 100 \times ( 1 - \frac{S ( t )}{S ( {t}_{peak} )} )$$

and wherein S(tₚₑₐₖ) represents the value of S(t) at the time at which a final peak is observed in the normalised data of said intensity as a function of time.

Optionally, the step of analysing said normalised data alternatively comprises calculating the percentage of the test meal which is remaining in the stomach of the subject as a function of time, R_{pc}, wherein:

$${R}_{pc} = 100 \times ( 1 - \frac{B ( t )}{{B}_{max}} )$$

and wherein B(t) represents the intensity contribution from the fluorescent contrast agent in the bloodstream of the subject as a function of time.

Optionally, the step of analysing said normalised data comprises calculating the percentage of the test meal which is remaining in the stomach of the subject as a function of time, R_{pc}, wherein:

$${R}_{pc} = 100 \times ( 1 - \frac{I ( t )}{I ( {t}_{peak 1} )} )$$

and wherein I(t) represents the normalised data of said intensity as a function of time; and
tₚₑₐₖ₁ represents the time at which the first peak is observed in said intensity as a function of time.

Optionally, any of the above methods according to the first aspect of the disclosure or the second aspect of the disclosure may be performed on said fluorescence intensity data without first normalising the fluorescence data. In other words, optionally, in the method for measuring gut permeability of a subject according to the first aspect of the disclosure, the step of normalising the fluorescence data to obtain normalised data of said intensity as a function of time may be omitted. Similarly, optionally, in the method for measuring gastric emptying rate according to the second aspect of the disclosure, the step of normalising the fluorescence data to obtain normalised data of said intensity as a function of time may be omitted. In either case, the data of said intensity as a function of time can be analysed as described above, without having been normalised before said analysis is performed.

### BRIEF DESCRIPTION OF THE DRAWINGS

The present disclosure may be carried out in various ways and embodiments of the disclosure will now be described by way of example with reference to the accompanying drawings in which:

FIG. 1 shows a flowchart illustrating a method for measuring gut permeability of a subject;

FIG. 2 shows the chemical structure of fluorescein sodium and its molecular weight (MW);

FIG. 3 shows exemplary data obtained using a method for measuring gut permeability of a subject and is a plot of fluorescence intensity versus wavelength for solutions comprising differing doses of fluorescein;

FIG. 4A shows a wearable probe being worn on a fingertip of a subject;

FIG. 4B shows a wearable probe being worn on a forearm of a subject;

FIG. 5A shows a mount for positioning the wearable probe of FIG. 4A in contact with the fingertip;

FIG. 5B shows a mount for positioning the wearable probe of FIG. 4B in contact with the forearm;

FIG. 6 shows an exemplary plot of fluorescence intensity data plotted against wavelength, for test measurements taken on a finger, arm and wrist of a test subject who was injected with fluorescein;

FIG. 7 shows confocal endomicroscopy images of the distribution of fluorescein measured at a location on an arm, a wrist and a finger of a subject after oral ingestion of fluorescein;

FIG. 8 shows a portable dual-channel fibre-optic fluorescence spectrometer optical system;

FIG. 9 shows the optical system of FIG. 8 mounted on a wheeled trolley;

FIG. 10 shows a plot of normalised fluorescence intensity as a function of time;

FIG. 11 shows two plots of normalised fluorescence intensity as a function of time recorded in the same subject on different days;

FIG. 12 shows a flowchart illustrating a method for measuring gastric emptying rate of a subject;

FIG. 13 shows the plot of FIG. 10, with the addition of an exemplary fitted curve which has been fitted to the normalised fluorescence intensity data;

FIG. 14 shows exemplary plots of normalised fluorescence intensity as a function of time, with exemplary fitted curves, and subplots showing residuals of the difference between the fluorescence data and the respective fitted curves;

FIG. 15A shows plots of normalised fluorescence intensity and paracetamol concentration as a function of time for data obtained using fluorescein and paracetamol based measurement methods respectively; and

FIG. 15B shows plots of percentage retention as a function of time based on the data shown in FIG. 15A.

### DETAILED DESCRIPTION

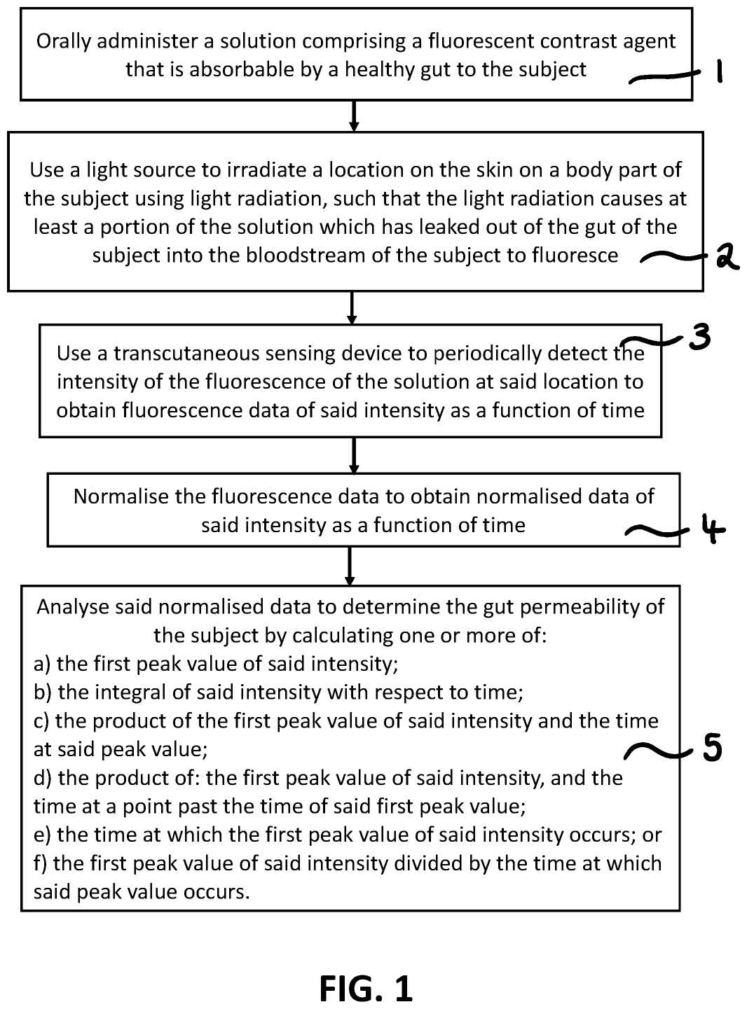

With reference to FIG. 1, the steps of a method for measuring gut permeability of a subject, and exemplary procedures for carrying out said method, shall now be described.

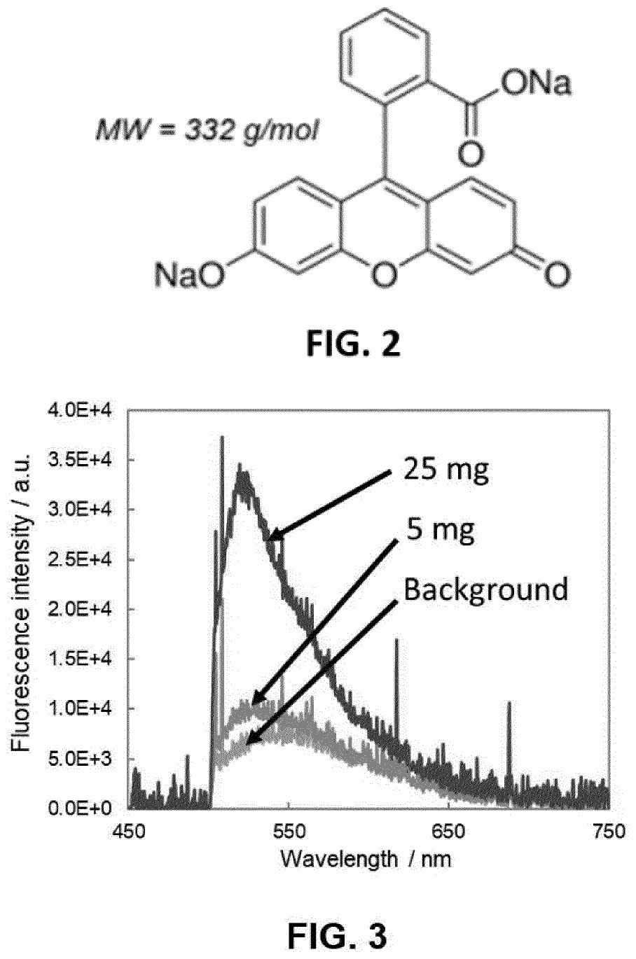

In a first step 1, a solution comprising a fluorescent contrast agent that is absorbable by a healthy gut is orally administered to the subject 13 (i.e. a patient). That is, the fluorescent contrast agent is delivered as a solution (in water or juice, for example), for the subject to drink. The chemical structure and molecular weight of an exemplary suitable contrast agent, fluorescein sodium, is shown in FIG. 2, which is a dye that is absorbable by a healthy gut.

Though, it is also envisaged that any other suitable fluorescent contrast agent may be used, for example fluorescein, methylene blue, fluorescein isothiocyanate conjugated dextran, or salts thereof, or combinations thereof.

In the example described below, the fluorescent contrast agent is fluorescein and is delivered as a dose of between 100 to 500 mg as a solution in 100 ml water. Though, it is also envisaged that the solution may comprise any other liquid instead of water, for example orange juice, or any other juice, and that any other dose of any other suitable fluorescent contrast agent and volume of the solution may be used. For example, a dose of 100 mg of methylene blue in 100 ml of orange juice may be used.

FIG. 3 shows exemplary data obtained using the method shown in FIG. 1 (the subsequent steps of which are outlined below) and shows a plot of fluorescence intensity (y axis) versus wavelength (x axis), for a solution comprising 5 mg of fluorescein as the fluorescent contrast agent (spectrum recorded 29 minutes after ingestion) and for a solution comprising 25 mg of fluorescein as the fluorescent contrast agent (spectrum recorded 37 minutes after ingestion). The plot shows that compared with the background spectrum, the fluorescence from 5 mg of fluorescein was only just about detectable, whereas the fluorescence from 25 mg of fluorescein was more clearly observable over the background spectrum. These results show that the limit of detection for fluorescein using the exemplary hardware described herein is below 25 mg. Preferably, to find an optimal balance between minimising the dose and maximising the signal-to-noise ratio, a dose of 500 mg of fluorescein may be used.

Referring back to FIG. 1, in a second step 2, a light source is used to irradiate a location on the skin on a body part of a subject using light radiation, such that the light radiation causes at least a portion of the solution which has leaked out of the gut of the subject into the bloodstream of the subject to fluoresce.

With further reference to FIG. 1, in a third step 3, a transcutaneous sensing device is used to periodically detect the intensity of the fluorescence of the solution at said location to obtain fluorescence data of said intensity as a function of time. In the example shown, said fluorescence data is spectral data. Though, it is also envisaged that said fluorescence data may have any other form, i.e. said fluorescence data need not necessarily be a spectrum for each time point (that is, for each periodic measurement obtained). For example, said fluorescence data could alternatively comprise a single intensity measurement for each time point (that is, for each periodic measurement obtained).

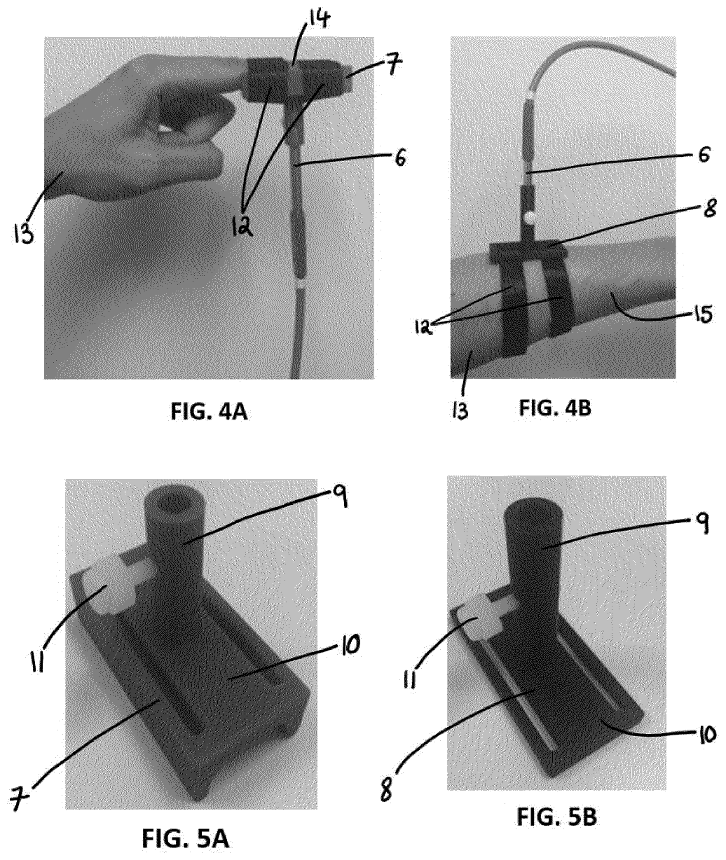

The light source and the transcutaneous sensing device are part of a wearable probe 6. As shown in FIGS. 4A and 4B respectively, the wearable probe 6 may be worn on a fingertip 14 or a forearm 15 of a subject 13. Though, it is also envisaged that the wearable probe 6 may be worn on or around any other body part, such as a finger, wrist, arm or earlobe. The body part should be chosen to optimise the signal obtained by the transcutaneous sensing device, because the thickness of the skin of the subject 13 may affect the signal, and the behaviour of the fluorescence signal with respect to time may be dependent on the body location. This may be due to leakage of the fluorescent contrast agent from blood vessels into the skin epithelium. As it is the concentration of the fluorescent contrast agent in the bloodstream that is of interest, a body location should be chosen where this effect of leakage into the epithelium is minimised. At the fingertip and the earlobe, the blood vessels are close to the surface of the skin, which means that the detected fluorescence signal is least affected by the residual dye that has leaked into the epithelium. If the wearable probe 6 is worn on a body part other than a fingertip or an earlobe (or if nonetheless despite wearing the probe on the fingertip or earlobe, the effect of some leakage is observed), correction in the data analysis to account for the epithelial leakage may be required.

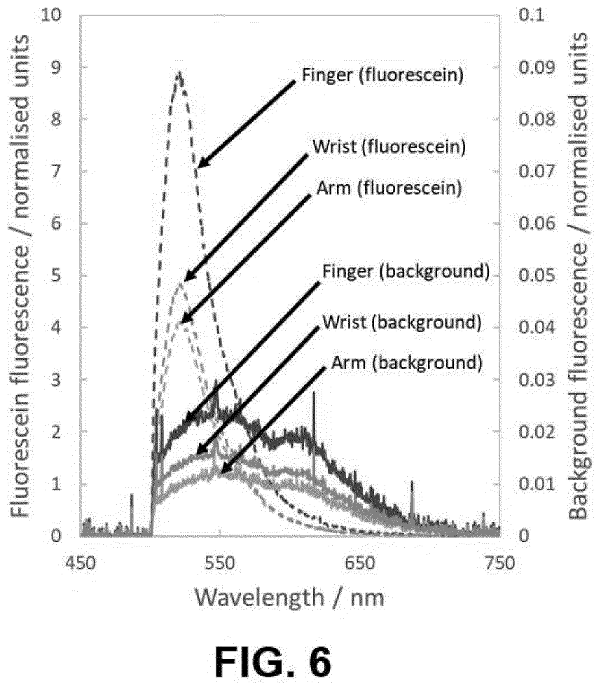

To illustrate how some body parts may be a more suitable measurement location than others, FIG. 6 shows an exemplary plot of fluorescence intensity data plotted against wavelength, for test measurements taken on a finger, arm and wrist of a test subject who was injected with fluorescein. The background fluorescence is also plotted. FIG. 6 shows that the fluorescence intensity may be greater at a finger than at an arm or a wrist, most likely due to the proximity of blood vessels to the surface of the skin at a finger, and that a stronger and more easily observable signal may thus be obtained on a finger, than on an arm or wrist.

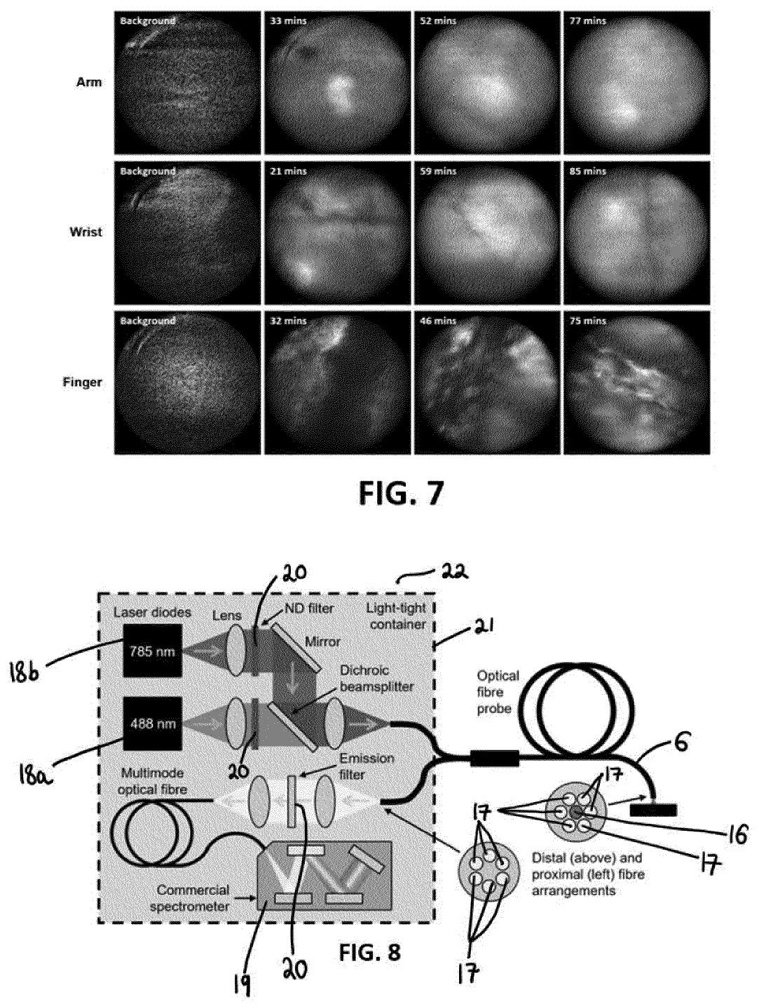

Further data illustrating that a finger may be a more preferable location than an arm or a wrist is shown in FIG. 7. FIG. 7 shows exemplary confocal endomicroscopy images of the distribution of fluorescein measured at a location on an arm, a wrist and a finger of a subject after oral ingestion of fluorescein. Labels are shown indicating the times in minutes after oral ingestion for the different confocal fluorescence endomicroscopy images. All of the images show a field of view with a 240 μm diameter. As shown in FIG. 7, compared with the background signal, only a diffuse fluorescence signal was detected when recording images at the arm and wrist, with no discernible structure observed. At the finger however, clear structure was observed with fluorescence appearing to emanate from cellular structures and/or blood vessels. This illustrates that the finger may be the optimal location for positioning the probe 6.

The wearable probe is configured to be positioned in contact with the body part by a mount 7, 8. Exemplary mounts 7 and 8 are shown in FIGS. 5A and 5B respectively and may be manufactured by additive manufacturing, for example. Each of the exemplary mounts 7, 8 comprises a cylindrical portion 9 for receiving an end portion of the wearable probe 6, a substantially planar portion 10 for being positioned proximate to the relevant body part, and an adjusting means 11 for securing the wearable probe 6 inside the cylindrical portion 9. The shape and/or dimensions of the mount 7, 8 may be chosen depending on the size and shape of the wearable probe 6 and depending on which body part the wearable probe 6 is to be worn on. As shown in FIGS. 4A and 4B, securing means 12 such as tape, straps, and/or hook and loop fasteners can be used to fix the mount 7, 8 and the wearable probe 6 to the relevant body part, 14, 15 of the subject 13. It is also envisaged that transcutaneous fluorescence measurements may alternatively be performed by manually holding the probe 6 in contact with the subject's 13 skin.

The light source, which may be a laser or LED for example, is chosen in one example described herein to be a laser having a centre wavelength in the range 450-490 nm. The transcutaneous sensing device is configured to detect both the backscattered excitation signal (i.e. the excitation power of the light source) and the fluorescence signal from the fluorescent contrast agent in the solution in the bloodstream of the subject 13, and this can be achieved, for example, by the use of two detectors with appropriate optical filters (which may be better suited to a smaller LED/photodiode-based sensor), or through the use of a single detector and a rotating filter wheel (which may be better suited for use with a laser or fibre-optic-based sensor as described in the example herein).

The transcutaneous sensing device is designed to allow measurement of the excitation and fluorescence signals at the same location on the subject 13 (for example, by placing the detectors in close proximity to one another or by using the same detector in combination with a rotating filter wheel). That is, it is envisaged that the transcutaneous sensing device may be configured to allow measurement of the excitation and fluorescence signals at the same location on the subject 13, or alternatively at different respective locations on the subject 13, the different respective locations being in close proximity to one another.

By measuring the excitation power at the same (or a very similar/nearby) location as the fluorescence signal, it can then be possible to normalise the fluorescence data to correct for factors including fluctuations in the excitation intensity, movement of the wearable probe, skin tone, skin absorption properties, and/or skin scattering properties etc. In turn, this can allow the detected fluorescence signal (corrected for excitation power) to be analysed quantitatively as a function of time. This is particularly important, as only a single (i.e. only one) fluorescent contrast agent is used in the method (fluorescein in the example described herein), so it is not possible to calculate ratios of intensities detected for two or more dyes (which can inherently correct for intensity fluctuations and the other issues highlighted above). Thus, appropriately normalising the fluorescence signal and recording data as a function of time allows for quantitative assessment of the fluorescence intensity and of the time taken for the fluorescent contrast agent to get into the bloodstream of the subject 13, both of which are of interest when calculating gut permeability and/or gastric emptying rate.

The excitation power (i.e. the backscattered excitation signal) can be measured by passing the light through an appropriate optical filter (i.e. a 500 nm short pass filter or a 10-20 nm bandpass filter centred on the excitation source wavelength), which acts to reject the fluorescence signal thereby allowing exclusive measurement of the excitation light. Alternatively, the excitation power can be measured simply by detecting unfiltered light—as the excitation power is orders of magnitude more intense than the fluorescence, a measurement of the unfiltered signal can act as a good approximation of the excitation light intensity. The fluorescence signal can then be measured by passing the light through a 500 nm long pass filter. This cuts out the excitation signal allowing sensitive detection of the fluorescein fluorescence.

A schematic diagram of an exemplary portable dual-channel, fibre-optic fluorescence spectrometer optical system 22 suitable for use in said second 2 and third 3 steps of the method is shown in FIG. 8. The insets of FIG. 8 show distal and proximal arrangements of optical fibres in the bifurcated fibre wearable probe 6, with excitation fibres 16 being shown in blue, and collection fibres 17 being shown in yellow. “ND” in FIG. 8 represents “neutral density”. The exemplary spectrometer comprises two laser sources 18a and 18b (at 488 nm and 785 nm respectively) for excitation of fluorescence, a commercial spectrometer 19, optical excitation/emission filters 20, an automated filter wheel (containing the emission filters), to ensure reliable detection of the fluorescence signals, and a bifurcated optical fibre wearable probe 6 to deliver light to and collect light from the subject's 13 skin.

In the example, laser sources at 488 nm and 785 nm were chosen to permit excitation of fluorescence from fluorescein (or fluorescein isothiocyanate) and ICG (indocyanine green) respectively (and from other dyes with comparable spectral properties), for experimental purposes. It is envisaged that the laser source(s) (and optical filters) may alternatively be chosen to have any other wavelength(s) as appropriate.

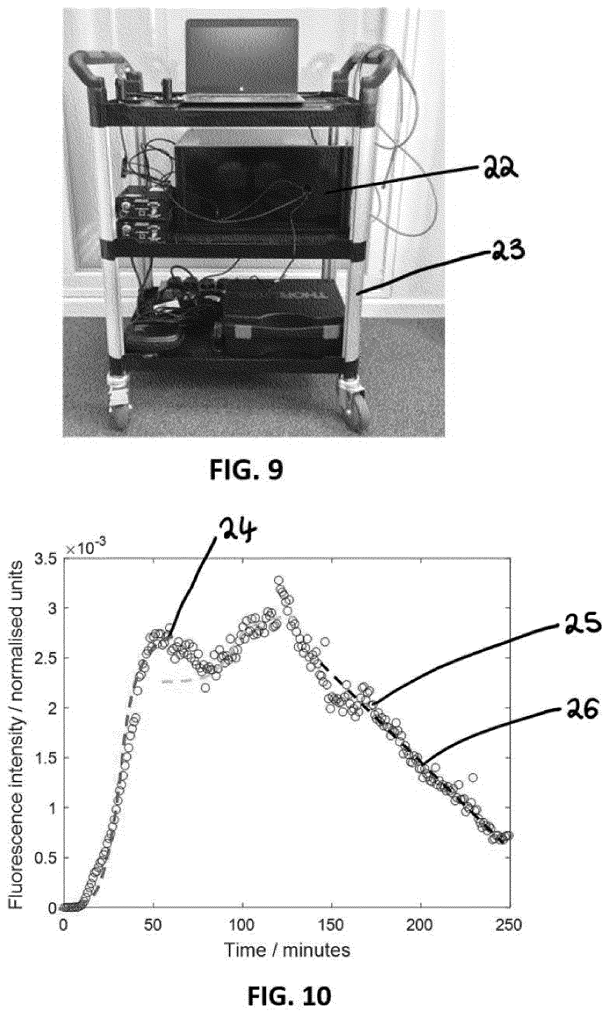

The optical system 22 is contained within an anodised light-tight aluminium box 21 for the purposes of laser safety and is controlled by a laptop computer running LabVIEW software. The entire optical system 22 can be mounted on a wheeled trolley 23 to allow use within clinics, as shown in FIG. 9.

For each individual measurement, the transcutaneous sensing device can be programmed to integrate until a suitable signal level is acquired. Typical acquisition times are in the range 100 ms-15 s. Programming the transcutaneous sensing device in this way can ensure that good signal levels are acquired at all time points (i.e. at all of the periodic measurements), even when the fluorescence levels are low (for example at the beginning or end of the measurement process). It can also ensure that there are no problems related to detector saturation when the fluorescence signal is at its highest. This can allow for measurements to be made reliably across subjects/patients with different skin tones without requiring higher doses in those with darker skin. Instead of requiring higher doses or higher excitation powers (which may not be clinically acceptable), the transcutaneous sensing device can automatically acquire data with longer integration times to allow for suitable signal levels to be obtained.

Referring back to FIG. 1, in a fourth step 4, the fluorescence data is normalised to obtain normalised data of said intensity as a function of time.

In the example, the fluorescence intensity value for each time point (i.e. for each periodic measurement) is normalised according to the acquisition time and excitation power used in each case, thereby allowing all time points to be compared against one another. This can allow measurements from different days/times or in different participants to be quantitatively compared. Furthermore, it means that the fluorescence values can be used to provide meaningful quantifications of variations in permeability (across different subjects/patients or over time in one subject/patient) rather than simply providing a binary assessment as to whether a contrast agent has passed the gut barrier.

In the example described herein, to achieve this normalisation, the background signal is first subtracted from the fluorescence signal for each time point. With spectrally resolved data (for example, as collected using a fibre-optic fluorescence spectrometer optical system 22 as shown in FIG. 8), the background is calculated as the average intensity over the wavelength range 350-450 nm (over which no signal is observed). The background-subtracted spectra are then summed over the wavelength range containing the spectral peak of the fluorescence signal (500-580 nm for fluorescein). This integrated fluorescence value is then normalised according to both integration time and laser power (by dividing by the product of the two). Excitation (laser) power values are also calculated based on excitation (laser) spectra (which are recorded immediately after/before each fluorescence spectrum). To obtain the laser power values, the laser spectra are background subtracted as described above and are then summed over the range 485-492 nm. These summed values are then normalised according to the integration time used to collect the laser spectra (typically 1 ms). Thus, the normalised integrated fluorescence intensity values for fluorescein can be calculated according to the equation:

$${I}_{norm} = \frac{( {\sum }_{λ = 500 nm}^{λ = 580 nm} ( I ( λ ) - {B}_{F} ) ) \times {t}_{L}}{( {\sum }_{λ = 485 nm}^{λ = 492 nm} ( L ( λ ) - {B}_{L} ) ) \times {t}_{F}}$$

Here, Iₙₒᵣₘ is the integrated, normalised fluorescence intensity, λ is the wavelength, t_{F} and t_{L} are the integration times for the fluorescence and laser spectra respectively, I(λ) is the fluorescence spectrum, L(λ) is the laser spectrum, and B_{F} and B_{L} represent the background values for the fluorescence and laser spectra respectively.

In the case that a wearable probe 6 based on LEDs and photodiodes is used (where the collected data is not spectrally resolved), the background can be measured by recording a measurement with the fluorescence detector with the excitation light (i.e. the light source) switched off. In addition, the excitation power can be measured using a second photodetector. This can be background subtracted by recording the signal level with the excitation LED switched off. The normalised fluorescence intensity can then be calculated as in the above previous equation but without any summations (as the wavelength ranges are defined by the optical filters placed in front of the photodetectors and the data is effectively summed over those ranges as it is collected). Thus, the normalised intensity can be written as:

$${I}_{norm} = \frac{( {I}_{F} - {B}_{F} ) \times {t}_{E}}{( {I}_{E} - {B}_{E} ) \times {t}_{F}}$$

where I_{F} and I_{E} respectively represent the fluorescence and excitation intensity values, B_{F} and B_{E} respectively represent the background values for the fluorescence and excitation light detectors, and t_{F} and t_{E} respectively represent the integration times used for the fluorescence and excitation light measurements. It is noted that if different integration times are used to record the fluorescence/excitation levels and the background levels then this needs to be factored into the above previous equation—i.e. B_{E} and B_{F} should be normalised according to their respective acquisition times. In this case, the above previous equation would become:

$${I}_{norm} = \frac{( \frac{{I}_{F}}{{t}_{F}} - \frac{{B}_{F}}{{t}_{BF}} )}{( \frac{{I}_{E}}{{t}_{E}} - \frac{{B}_{E}}{{t}_{BE}} )}$$

Where t_{BF} and t_{BE} respectively represent the integration times used to record the background measurements for the fluorescence and excitation light detectors.

The above normalisation procedure can be performed on the fibre-optic fluorescence spectrometer optical system 22, meaning that the output data is immediately usable for diagnostic purposes without further analysis.

Following collection and normalisation of the fluorescence data, a plot of normalised fluorescence intensity as a function of time (I(t)) can be provided. A typical example of such data is shown in FIG. 10. Due to the normalisation procedure described above, such a plot can be used to quantify gut permeability in a number of ways.

That is, referring back to FIG. 1, in a fifth step 5, the normalised data is analysed to determine the gut permeability of the subject by calculating one or more of a) the first peak value of said intensity; b) the integral of said intensity with respect to time; c) the product of the first peak value of said intensity and the time at said peak value; d) the product of: the first peak value of said intensity, and the time at a point past the time of said first peak value; e) the time at which the first peak value of said intensity occurs; or f) the first peak value of said intensity divided by the time at which said peak value occurs. It should be noted that although in the example described herein the analysis is performed on normalised data, it is also envisaged that the analysis could alternatively be performed on non-normalised data, if a decrease in the accuracy and reliability of the analysis could be accepted. Such analysis may still provide useful diagnostic information. Specifically, this would be achieved by amending the above previous equations for Iₙₒᵣₘ to read as

$${I}_{int} = {\sum }_{λ = 500 nm}^{λ = 580 nm} ( I ( λ ) - {B}_{F} )$$

$$and$$

$${I}_{int} = {I}_{F} - {B}_{F}$$

where Iᵢₙₜ represents the integrated, non-normalised fluorescence intensity data for each time point for spectral (first equation) and non-spectral data (second equation) respectively. In the description below, normalised data is used by performing all further analysis on data normalised according to the first Iₙₒᵣₘ equation above. However, identical analysis could be performed on non-normalised data by starting with either of the two above previous equations for lint.

Continuing with analysis on normalised data, as an example, the intensity of the first peak 24 on the fluorescence versus time curve 25 (see FIG. 10) can be used to provide a readout of the peak concentration of the fluorescent contrast agent in the solution in the bloodstream of the subject 13. The first peak 24 represents the point in time at which there is a maximum concentration of the fluorescent contrast agent in the bloodstream and typically occurs before significant elimination from the body of the subject 13 has occurred (for example, via the kidneys or liver).

In some cases, the fluorescence signal may continue to increase after the initial peak 24 due to leakage of the fluorescent contrast agent from the bloodstream into the epidermis of the skin at the measurement location. Thus, to accurately identify the initial peak 24 (which represents the peak concentration of the fluorescent contrast agent in the bloodstream of the subject 13), it is necessary to collect data with sufficient temporal resolution. Recording measurements once every minute may comfortably ensure that this is the case. Conversely, using measurement intervals of over five minutes may risk losing the resolution required to observe the initial peak 24.

Alternatively, to account for the effects of gastric emptying rate (which may alter the time and intensity of the initial peak 24), gut permeability can also be quantified by calculating the area under the curve 25 up to the initial peak 24, which may be determined using mathematical integration. This provides a measure of the total amount of the fluorescent contrast agent that has leaked from the gut into the bloodstream up to the time of the initial peak 24. This approach is analogous to measurement of total lactulose recovery used in many sugar-based urine permeability assays. However, it does not suffer from the issue of not knowing the exact time at which to collect urine—by identifying the time of peak concentration from the data it is possible to make an accurate quantification of the total recovery of fluorescein (or another fluorescent contrast agent of choice) in the bloodstream.

Alternatively, gut permeability can also be quantified by calculating the product of the peak intensity and the time at which the peak 24 occurs (or alternatively, by calculating the product of: the peak value of the intensity, and the time at a point past the time of the first peak value, as discussed below). This represents a simplification of the area-under-the-curve approach described above.

Thus, gut permeability can be quantified based on the normalised fluorescence data using one of the three equations below:

$${GP}_{1} = I ( {t}_{peak} )$$

$${GP}_{2} = {\sum }_{t = 0}^{{t}_{peak}} I ( t )$$

$${GP}_{3} = I ( {t}_{peak} ) \times {t}_{peak}$$

I represents the normalised fluorescence intensity (as calculated using the equations for Iₙₒᵣₘ above), t represents time, tₚₑₐₖₖ represents the time of the initial peak in the fluorescence data, and GP₁₋₃ represent three proposed quantifiers of gut permeability. (As an aside, it is also worth noting that I could alternatively represent the non-normalised fluorescence intensity data—i.e. as calculated using the equations for Iᵢₙₜ above—if a reduction in the reliability of the analysis can be tolerated). Alternatively, the equations for GP₂ and GP₃ may be modified as:

$${GP}_{2} = {\sum }_{t = 0}^{{t}_{peak +}} I ( t ) \\ {GP}_{3} = I ( {t}_{peak} ) ⨯ {t}_{peak +}$$

In these modified equations for GP₂ and GP₃, tₚₑₐₖ₊ represents a chosen time point after the first peak in the fluorescence data, for example a chosen time point equal to the sum of the value of time at which the initial peak in the fluorescence data occurs, plus a chosen percentage of said value of time.

In addition to the quantifiers presented in the above three equations, the time of the initial peak (tₚₑₐₖ) may also provide an indication of permeability, with lower tₚₑₐₖ values corresponding to higher permeabilities.

Furthermore, dividing I(tₚₑₐₖ) by tₚₑₐₖ will provide another quantifier that will vary with permeability, in this case increasing as a function of increasing permeability. Hence, two further permeability quantifiers can be defined as described below:

$${GP}_{4} = {t}_{peak} \\ {GP}_{5} = \frac{I ( {t}_{peak} )}{{t}_{peak}}$$

GP₄ and GP₅ may be confounded by the effects of gastric emptying rate, as higher emptying rates are also likely to produce changes in the values of GP₄ and GP₅. Conversely, GP₂ and GP₃ may provide a degree of correction for gastric emptying rates by multiplying by tₚₑₐₖ or by calculating the area under the curve up until tₚₑₐₖ. Thus, they may offer a more quantitative assessment of permeability.

Overall, it is possible to quantify permeability based on the normalised fluorescence intensity. This allows for quantification of the leakage of fluorescent contrast agents that pass even the healthy gut barrier and means that it will be possible to provide meaningful clinical data in a wide range of subjects/patients and not just those with extremely permeable guts. In addition, the quantifiers described above will provide measurements that vary in a continuous manner with respect to permeability rather than simple binary markers that provide only coarse clinical outcomes of whether a gut is ‘permeable’ or ‘not permeable’. This will permit monitoring of patients' responses to therapies and other interventions (for example, nutritional interventions) and may also allow comparison of values measured in different individuals (i.e. for screening/diagnostic applications). This is possible due to the normalisation procedure described above and due to the collection of data with time resolution sufficient to allow identification of the initial peak in the fluorescence versus time curve (i.e. the point of peak concentration in the blood).

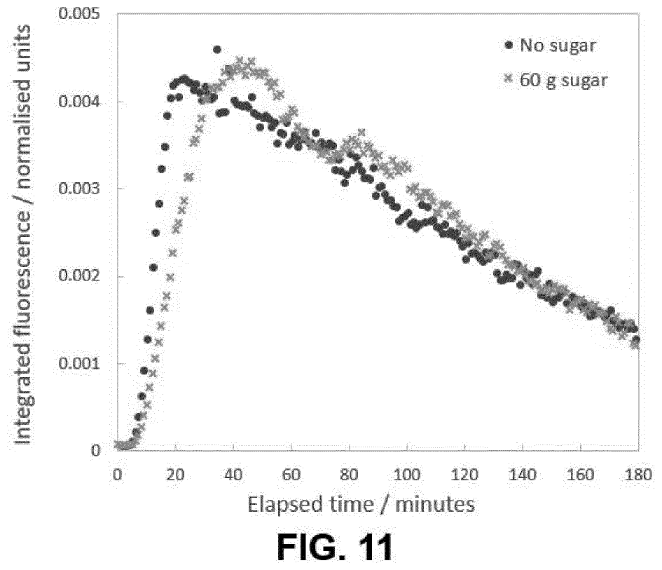

Finally, in reference to said method for measuring gut permeability, FIG. 11 shows a comparison of data acquired with and without a hyperosmotic solution, the exemplary hyperosmotic solution used containing 60 g of sugar. The plot shows the normalised fluorescence intensity plotted against the elapsed time in minutes, and results from an experiment in which a participant took two gut permeability tests on separate days. In the first case, the solution consumed consisted of 500 mg fluorescein dissolved in 100 ml water. In the second experiment, the solution consisted of 500 mg fluorescein and 60 g sugar dissolved in 100 ml water. The concentrated sugar solution is known to have a hyperosmotic effect—i.e. it drives a temporary increase in intestinal permeability. The exemplary experimental data shown in FIG. 11 thus shows the ability to assess changes in gut permeability using a single fluorescent contrast agent (e.g. fluorescein) as described above. As shown in FIG. 11, when 60 g sugar was included in the solution, the peak value (GP₁) was fractionally higher compared to the no sugar experiment. Furthermore, large increases were observed in the area under the curve (GP₂) and the value of I(t)×tₚₑₐₖ (GP₃). Accordingly, the exemplary data shown in FIG. 11 provides a preliminary validation of the proposed permeability markers GP₁-GP₃ described above.

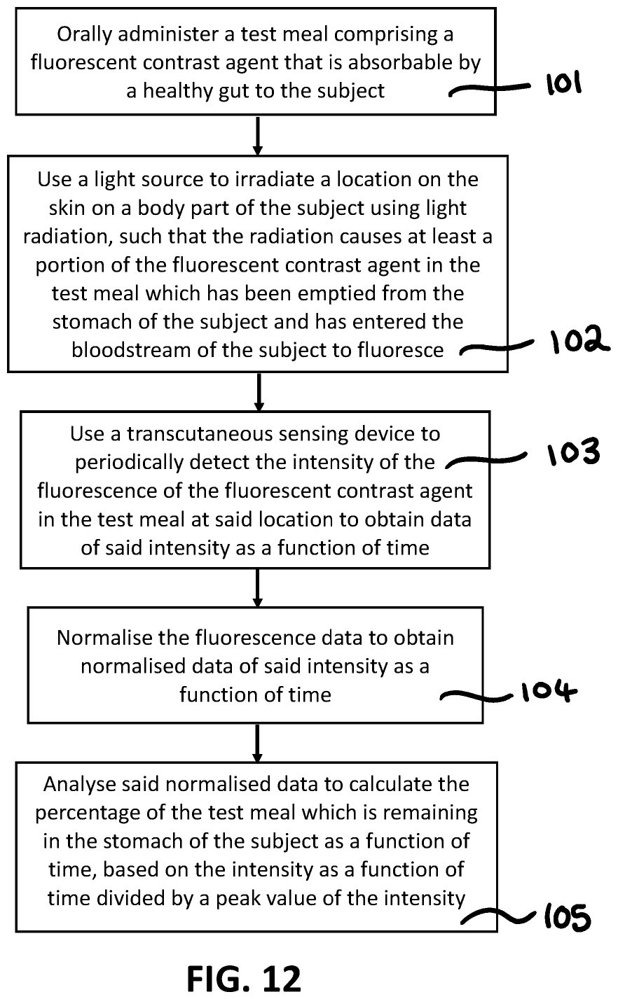

Some of the aspects of the above method and procedures for measuring gut permeability may also be used in a method for measuring gastric emptying rate. With reference to FIG. 12, the steps of a method for measuring gastric emptying rate (i.e. how quickly the stomach is emptied) of a subject, and exemplary procedures for carrying out said method, shall now be described.

Steps 101 to 104 of the method shown in FIG. 12 are similar to the steps 1 to 4 respectively of the method shown in FIG. 1 and thus may be performed in a substantially similar manner and using substantially similar hardware as steps 1 to 4 described above.

Step 101 of the method shown in FIG. 12 differs from step 1 of the method shown in FIG. 1 and described above in that to allow for measurement of gastric emptying rate in a realistic and physiologically interesting situation, the fluorescent contrast agent is prepared as part of a liquid test meal or a solid test meal (rather than in water or juice). This is because a liquid test meal or a solid test meal will empty more slowly from the stomach than pure water or juice. A suitable exemplary liquid test meal is a milkshake, and a suitable exemplary solid test meal is scrambled eggs on toast. Though, it is also envisaged that any other suitable liquid test meal or solid test meal may be employed in the test meal.

Advantageously, a milkshake may be designed to represent a liquid-based test meal that empties from the stomach in a similar manner to solid food (which is important physiologically) and to be comfortable/enjoyable for the subject 13 to consume. 500 mg fluorescein can be used in an exemplary milkshake formulation, which is the clinically approved dose of fluorescein for intravenous injection and is included as it gives optimal signal levels (as it is the highest clinically acceptable dose). However, lower doses can also be used (down to approximately 100 mg) while retaining acceptable signal-to-noise ratios. This may allow for reduction of the dose of contrast agent and, hence, reduction of the impact and risk placed on the subject 13. An exemplary milkshake may also comprise chocolate powder, protein powder, sugar and/or milk.

Prior to consuming the test meal, the subject 13 may be asked to fast for 12 hours, i.e. they may be asked to not consume any food or drink (with the possible exception of water) before the method for measuring gastric emptying rate is performed on them. This can be achieved by performing measurements in the morning and asking the subject 13 to fast overnight prior to the measurements. This can ensure that the gastric emptying rate measurement is not adversely affected by any residual food or drink in the stomach or intestine of the subject 13.

At the beginning of the measurement process, the transcutaneous sensing device is attached to the skin of the subject 13 at the chosen location (for example, a fingertip). Fluorescence measurements then begin before ingestion of the contrast agent to permit measurement of a baseline/background signal. In the example described here, the fluorescence signal and excitation power are measured using the transcutaneous sensing device once every minute. Though, it is also envisaged that alternative timings (i.e. measurement frequencies) can also be used, but one measurement per minute advantageously allows for collection of data with good signal-to-noise ratio and with sufficient temporal resolution.

After the first measurement has been made, the subject/patient is asked to consume the milkshake as quickly as possible (for example, over the following three minutes). A consumption time of three minutes can help ensure that the entire test meal (milkshake) reaches the stomach quickly but also means that the subject/patient can consume the drink comfortably. Fluorescence (and excitation power) measurements are then performed once per minute for the following four hours. This timescale ensures that all of the fluorescent contrast agent has emptied from the stomach in the majority of subjects and that at least one hour's worth of data is collected during which no more of the fluorescent contrast agent is entering the bloodstream as a result of gastric emptying (i.e. the florescent contrast agent is simply being cleared from the bloodstream). This is referred to as steady state clearance. Measuring the steady state clearance rate can allow for the accurate conversion of the fluorescence data into a measurement of percentage of stomach contents emptied. Though, it is also envisaged that different measurement timescales may also be used. For example, fluorescence (and excitation power) measurements may be performed for the following thirty minutes, rather than for the following four hours. The measurement timescale may be chosen based on the calculation used. Advantageously, short measurement timescales can allow for the results to be reported more quickly, which will reduce the impact on the subject 13.

For each individual measurement, the transcutaneous sensing device is programmed to integrate until a suitable signal level is acquired. Typical acquisition times are in the range 100 ms-5 s. Programming the transcutaneous sensing device in this way can ensure that good signal levels are acquired at all time points, even when the fluorescence levels are low (for example at the beginning or end of the measurement process). It can also ensure that there are no problems related to detector saturation when the fluorescence signal is at its highest. The fluorescence intensity value for each time point is normalised according to the acquisition time used in each case, thereby allowing all time points to be compared against one another.

The resulting data consists of the fluorescein fluorescence intensity (normalised according to excitation power and acquisition time) as a function of time. This can be used to calculate gastric emptying rate in a fifth step 105 of the method, as described below.

The method of FIG. 12 differs from the method of FIG. 1 in that in a fifth step 105, rather than analysing the normalised data to determine the gut permeability of the subject, the normalised data is instead analysed to calculate the percentage of the test meal which is remaining in the stomach of the subject as a function of time. This is achieved by calculation as a function of (i.e. based on/depending from) the intensity as a function of time divided by a peak value of the intensity. This calculation can be performed as outlined below. It should be noted that although in the example described herein the analysis is performed on normalised data, it is also envisaged that the analysis could alternatively be performed on non-normalised data, if a decrease in the accuracy and reliability of the analysis could be accepted. Such analysis may still provide useful diagnostic information. In either case, prior to the analysis described below, the intensity data is prepared according to the equations for Iₙₒᵣₘ (for normalised data) or Iᵢₙₜ (for non-normalised data) as outlined above in the discussion of measurement of gut permeability.

A typical fluorescence versus time curve 25 has a shape as shown in the example of FIG. 10. In the example shown, initially, the fluorescence signal increases as a function of time with a sigmoid-like shape (as shown by the (red) dashed line which begins at the point (0,0)) until an initial peak 24 is reached. Following the initial peak 24, a slower increase (or in some cases a plateau) is observed (indicated by the (yellow) second dashed line) prior to a steady decrease 26 as the fluorescent contrast agent is eliminated from the body.

The initial increase predominantly represents the uptake of the fluorescent contrast agent from the gut into the bloodstream of the subject 13. The secondary increase is thought to be due to the leakage of the fluorescent contrast agent from blood vessels into the epithelium (which leads to an increase in the observed fluorescence intensity as the contrast agent gets closer to the sensing region of the probe). As transcutaneous fluorescence spectroscopy does not provide measurements of the absolute concentrations of the fluorescent contrast agent in the blood and as the relative intensities of the fluorescent contrast agent in the blood and the fluorescent contrast agent in the epithelium are not known, it can be challenging to directly and accurately calculate percentage retention curves (i.e. the percentage of the contrast agent remaining in the stomach as a function of time) from the fluorescence data as is typically performed for paracetamol absorption tests or breath tests. Accordingly, a suitable exemplary approach based on least squares fitting is presented below, which allows the percentage retention curve to be extracted from the fluorescence data.

The fluorescence versus time curve 25 can be described by the sum of two sigmoid (logistic) functions—representing the uptake of the fluorescent contrast agent into the bloodstream (as shown by the (red) dashed line which begins at the point (0,0) in FIG. 10) and the leakage from blood vessels into the epithelium (indicated by the (yellow) second dashed line in FIG. 10) respectively—and a linearly decreasing term that represents the elimination of the fluorescent contrast agent from the body of the subject 13, as shown in the exemplary plot of FIG. 10. Thus, the fluorescence intensity as a function of time, I(t), can be written as:

I(t)=B(t)+L(t)−E(t)

where B(t) represents the intensity contribution from the fluorescent contrast agent in the bloodstream as a function of time, L(t) represents the intensity contribution from the fluorescent contrast agent that has leaked into the epithelium as a function of time, and E(t) represents the intensity contribution from the fluorescent contrast agent that has been eliminated from the body (and therefore does not contribute to the total intensity) at time t. These functions can be defined as shown below:

$$B ( t ) = \frac{{B}_{max}}{1 + exp ( - {k}_{B} ( t - {t}_{B \frac{1}{2}} ) )} \\ L ( t ) = \frac{{L}_{max}}{1 + exp ( - {k}_{L} ( t - {t}_{L \frac{1}{2}} ) )} \\ E ( t ) = ( B ( t ) + L ( t ) ) Ct$$

Bₘₐₓ represents the maximum intensity contribution from the fluorescent contrast agent in the bloodstream, while k_{B} and t_{B1/2} respectively represent the logistic growth rate (steepness) of the B(t) curve and the time at which B(t) reaches half of its maximum value. Similarly, Lₘₐₓ represents the maximum intensity contribution from the fluorescent contrast agent in the epithelium while k_{L} and t_{L1/2} represent the logistic growth rate and half-maximum time point for the L(t) curve. Lₘₐₓ and Bₘₐₓ determine the relative levels of fluorescence observed from the fluorescent contrast agent in the blood and the fluorescent contrast agent in the epithelium. As this approach involves fitting of these parameters, prior knowledge of these relative levels is advantageously not required. C represents the clearance rate—the rate at which the fluorescent contrast agent is eliminated from the body. The intensity contribution from the total amount of the fluorescent contrast agent eliminated by time t (E(t)) is then defined as the product of the time (t), the clearance rate (C) and the total intensity contribution from the fluorescent contrast agent in the system at that time point (B(t)+L(t)).

By substitution, the following expression for I(t) can thus be obtained:

$$I ( t ) = ( 1 - Ct ) ( \frac{{B}_{max}}{1 + exp ( - {k}_{B} ( t - {t}_{B \frac{1}{2}} ) )} + \frac{{L}_{max}}{1 + exp ( - {k}_{L} ( t - {t}_{L \frac{1}{2}} ) )} )$$

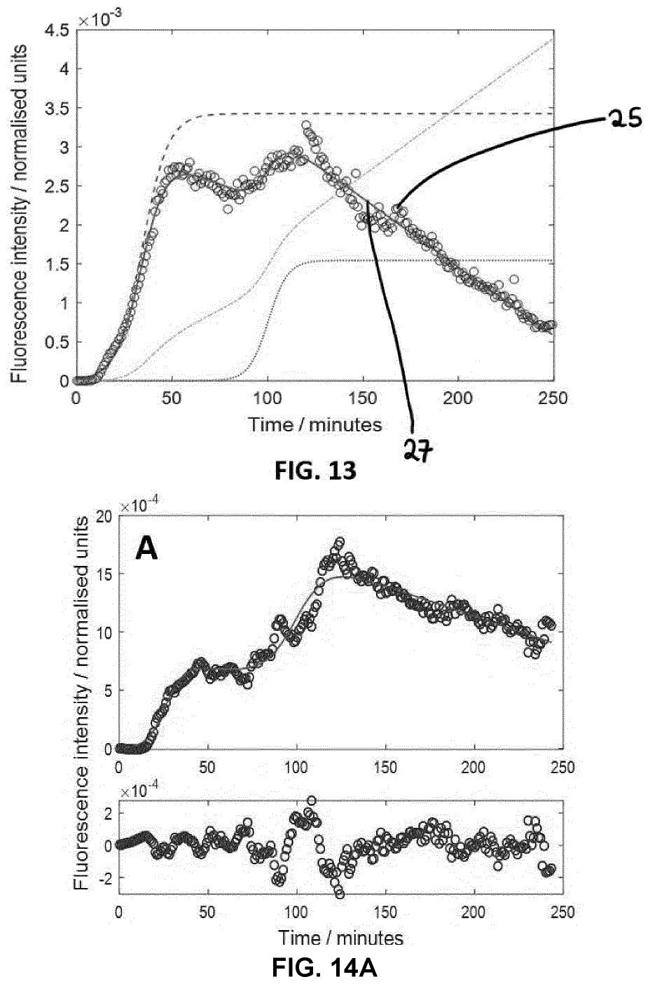

This equation can now be fit to the observed I(t) data using least squares fitting, for example, or another numerical fitting procedure, in order to extract the parameters C, Bₘₐₓ, Lₘₐₓ, k_{B}, k_{L}, t_{B1/2} and t_{L1/2}. This can be achieved using a range of least squares fitting algorithms (and using a variety programming languages), for example, using the ‘Isqcurvefit’ function in MATLAB. As data is collected at approximately one minute intervals in the example described herein, there are many more data points than unknown values, meaning that fitting can be performed accurately. An exemplary fitted curve 27 is shown in FIG. 13, for the exemplary data shown in FIG. 10.

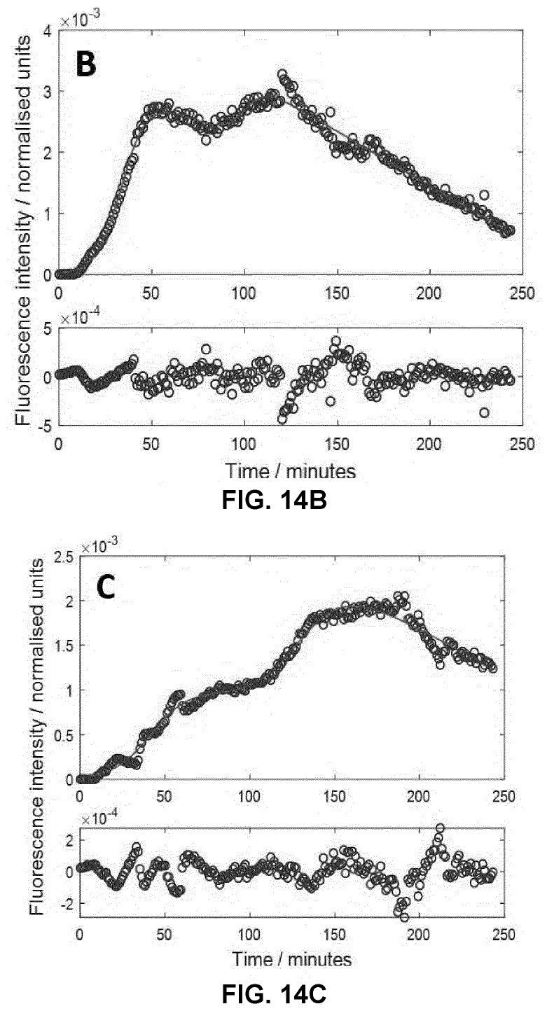

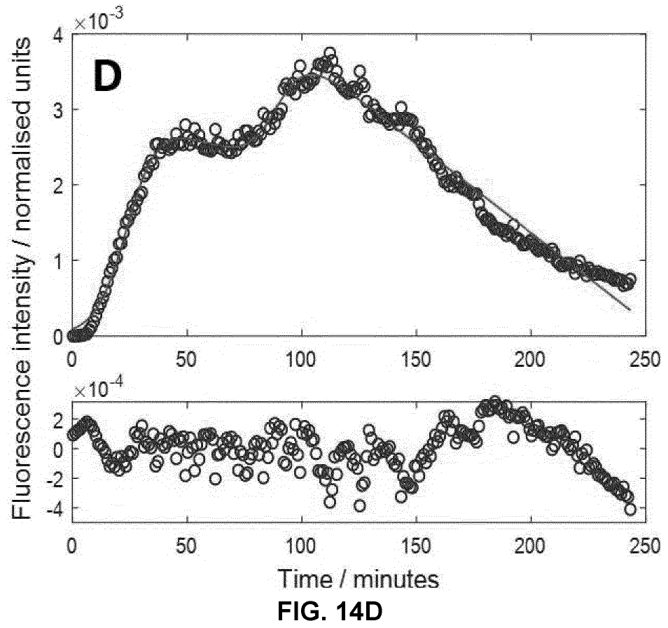

Further examples of other fitted curves of fluorescence versus time are shown in FIG. 14, which includes exemplary plots A, B, C and D. The exemplary curves shown in FIG. 14 have been fitted according to the method described above. In each of the examples, A, B, C and D, there is shown a main plot (top) and a subplot (bottom). In each of the main plots, the fluorescence versus time data points are represented by a plurality of circular markers, and the solid lines represent the corresponding fitted curve which has been fitted according to the method described above. The circular markers in each of the corresponding subplots represent the residuals—i.e. each data point/circular marker in the subplots represents the difference between the actual data point (represented by the corresponding circular marker in the main plot) and the fitted curve (represented by the solid line in the main plot) at each data point/time value. As can be understood by examining each of the main plots with its corresponding subplot, for examples A, B, C and D in FIG. 14, good fits are obtained in all four examples. This illustrates success of the aforementioned fitting method.

Following the fitting procedure, the fitted parameters can be used to define the amount of the fluorescent contrast agent that has emptied from the stomach at time t, S(t). Assuming that S(t) can be approximated as the sum of the intensity contribution from the fluorescent contrast agent in blood vessels and the amount of the fluorescent contrast agent eliminated by that time point, S(t) can be written as:

S(t)=B(t)+E_{B}(t)

where E_{B}(t) represents the elimination of the fluorescent contrast agent from blood vessels and is defined as:

E_{B}(t)=B(t)Ct

This is based on the assumption that the leakage of the fluorescent contrast agent from blood vessels into the epithelium may have a negligible effect on the absolute value of S(t). This is a sound assumption as L(t) typically entails a much slower logistic growth than B(t) and does not have a significant effect on the observed I(t) data until later in the time course, by which time the stomach is expected to have largely emptied.

It is then possible to calculate the percentage of the fluorescent contrast agent retained in the stomach, R_{pc}, at time t:

$${R}_{p c} = 100 ⨯ ( 1 - \frac{S ( t )}{S ( {t}_{peak} )} )$$

S(tₚₑₐₖ) is the value of S(t) at the time point at which the final peak is observed in the I(t) data. It may be chosen to normalise the R_{pc} curve at this point, as this is the final time point prior to the steady state elimination phase (where the fluorescent contrast agent is being eliminated from the body and no further increases are observed in the fluorescence intensity). Thus, after tₚₑₐₖ, no further fluorescent contrast agent is emptying from the stomach.

If only a single peak is observed, then tₚₑₐₖ can be set as the beginning of the steady state elimination phase, in which a constant linear decrease in the fluorescence signal is observed. It can be easier to identify this point in the fluorescence data (where data points are collected approximately once every minute) than it is in paracetamol or breath tests (where longer measurement intervals of 10-15 minutes are typically used). In addition, the transcutaneous sensing device could be programmed to automatically end data acquisition once this point is reached (for example, once data over a chosen time interval has shown a constant linear decrease with no subsequent increase).

If it is not possible to accurately measure the elimination rate (for example, if a short acquisition was used), then the effect of elimination can be ignored and R_{pc} can be calculated based solely on the data in B(t). In this case, R_{pc} can be written as:

$${R}_{p c} = 100 ⨯ ( 1 - \frac{B ( t )}{{B}_{max}} )$$

Using the above approach, R_{pc} values can be obtained which are in good agreement with those obtained from paracetamol absorption tests. Thus, advantageously, this represents a method that can provide readouts with clear clinical value in the assessment of gastric emptying rate. For example, the calculated R_{pc} curves can be used to determine the times at which the percentage of the fluorescent contrast agent/test meal retained in the stomach has dropped to 75%, 50%, 25%, etc. of its initial value.

Furthermore, the extracted parameters (for example, Bₘₐₓ, t_{B1/2}, etc.) can also be used to quantify gut permeability in an analogous manner to that presented above (i.e. see the equations for GP₁-GP₅ above).

In conclusion, this approach involves fitting a function (based on two sigmoid terms and a linearly decreasing term) to the observed transcutaneous fluorescence data. As the approach fits the function to the data rather than performing direct calculations, it does not require prior knowledge of the relative intensity contributions of the fluorescent contrast agent in blood vessels and in the epithelium. Nor does it require knowledge of factors such as body weight, total volume of bodily fluid, etc., which are required to assess gastric emptying rate using paracetamol absorption tests (and are often estimated based on assumptions). Moreover, while the observed fluorescence trends may vary depending on the geometry of the transcutaneous sensing device/probe used to collect the data, this fitting-based approach will be immune to variations of this manner. For example, if probe geometry is adjusted to reduce sensitivity to L(t) (fluorescent contrast agent leakage into the skin's epithelium), the fitting will simply correct for this by minimising the value of Lₘₐₓ (or setting it to zero). Thus, this approach can be suitable for use with any transcutaneous fluorescence probe or transcutaneous sensing device design. Overall, it permits the extraction of parameters for assessment of gastric emptying rate (and gut permeability) that are suitable for clinical use. This is possible based on non-invasive data collection, without the need to collect blood, urine or other samples. In addition, analysis can be performed in real-time in an automated fashion with processing carried out by the transcutaneous sensing device itself, and the fitting-based approach may also allow for shorter acquisition times by providing good fits without the need to collect full time courses over more than four hours (which is typically required for gastric emptying rate analysis using paracetamol absorption tests and/or Carbon-13 (“13C”) breath tests).

As an alternative to the above fitting-based approach, percentage retention curves can also be calculated directly from the observed fluorescence data—i.e. directly from I(t). In this case, the percentage retention is defined as:

$${R}_{{pc}_{-} direct} = 100 ⨯ ( 1 - \frac{I ( t )}{I ( {t}_{peak 1} )} )$$

Here R_{pc_direct} is the percentage retention curve and tₚₑₐₖ₁ represents the time at which the first peak is observed in the fluorescence data (I(t)). The first peak is used for normalisation of the percentage retention curve in this case in order to allow rapid calculation. Using this approach, the transcutaneous sensing device can automatically detect the first maximum in the fluorescence versus time data and then immediately calculate the percentage retention curve (from which diagnostic parameters can be extracted—for example, the time at which the percentage retention drops to 75%, 50%, 25%, etc.).

Using this approach, diagnostic parameters can be reported within approximately 60 minutes, or more quickly, and the data obtained can show good agreement with paracetamol absorption tests, particularly for values of R_{pc_direct} in the range 50-100%. In addition, the direct calculation can minimise any potential for errors or inconsistencies in the analysis procedure.

To obtain the most accurate calculation of the percentage retention, a background subtraction can be performed on I(t) prior to its use in the above equation. This can be achieved, for example, by setting the background level as the first intensity value—i.e. I(t=0)—and subtracting this from each I(t) value. Alternatively, the background value can be defined as the average of, for example, the intensities recorded at the first 5 time points. With this approach, the exact time window chosen for background subtraction will be determined by the point at which the fluorescence versus time curve (I(t)) begins to increase (with the background region being defined as all points before the start of the initial increase). To ensure optimal results, this background subtraction should also be performed prior to the fitting process described above.

If it is possible to calculate the elimination, then this can also be included in the percentage retention calculation to further improve the accuracy of the calculation. For simplicity, in this example, the amount of the fluorescent contrast agent emptied from the stomach, S_{direct}(t), can be defined as:

S_{direct}(t)=I(t)+E_{I}(t)

and the elimination of the fluorescence signal, E_{I}(t), can be defined as:

E_{I}(t)=I(t)Ct

As above, C represents the clearance rate. This can be obtained via fitting (as described above) or via direct calculation based on data in the steady state elimination region of the fluorescence versus time curve. In the latter case, C can be defined as:

$$C = \frac{I ( {t}_{s 1} ) - I ( {t}_{s f} )}{\overline{I ( {t}_{s} )} ⨯ Δ {t}_{s}}$$

tₛ represents the time region over which steady state elimination is observed, tₛ₁ is the first time point in the steady state elimination region, t_{sf} is the final time point in the steady state elimination region, Δtₛ represents the change in time across the steady state interval (i.e. t_{sf}−t_{sf}), and I̅(̅t̅ₛ̅)̅ represents the mean fluorescence intensity over the steady state region. Having calculated C, E_{I}(t) and S_{direct}(t) as described above, the percentage retention curve can be calculated as:

$${R}_{{pc}_{-} direct} = 100 ⨯ ( 1 - \frac{{S}_{direct} ( t )}{{S}_{direct} ( {t}_{peak} )} )$$

In this case, tₚₑₐₖ can be chosen as the time point at which the first or second peak in the fluorescence data is observed, or the time point at which steady state elimination begins (for example, at a time point 5%, 10% or 20% past the time point at which said peak is observed), and this can be determined based on the data (for example depending on whether enough data is collected to observe the steady state region). This approach can also provide good agreement with paracetamol absorption test data. However, as this approach requires collection of data up until at least the beginning of the steady state region (to permit calculation/fitting of the clearance rate, C), it may necessitate longer acquisition times than direct calculation based on the equation

$${R}_{{pc}_{-} direct} = 100 ⨯ ( 1 - \frac{I ( t )}{I ( {t}_{peak 1} )} ) .$$

Overall, direct calculation of percentage retention curves/values based on the fluorescence versus time curves can provide accurate assessments that agree with gold standard data (i.e. paracetamol absorption tests), particularly for percentage retention values in the range 50-100%. Using the approach presented in the equation

$${R}_{{pc}_{-} direct} = 100 ⨯ ( 1 - \frac{I ( t )}{I ( {t}_{peak 1} )} ) ,$$

percentage retention values and curves can advantageously be calculated based on very short data collections (for example over 30-60 minutes). These calculations can be performed automatically by the transcutaneous sensing device allowing diagnostic parameters (for example time to 50% retention, etc.) to be reported much more quickly than with other approaches (for example paracetamol absorption tests require more than four hours of data collection plus processing of blood samples in a pathology lab and manual analysis of the resulting data, meaning that diagnostic results are rarely reported in less than 1-2 days). Hence, both of the above approaches for calculating the gastric emptying rate provide potential for significant improvements on the current standard of care along with considerably faster reporting of results.

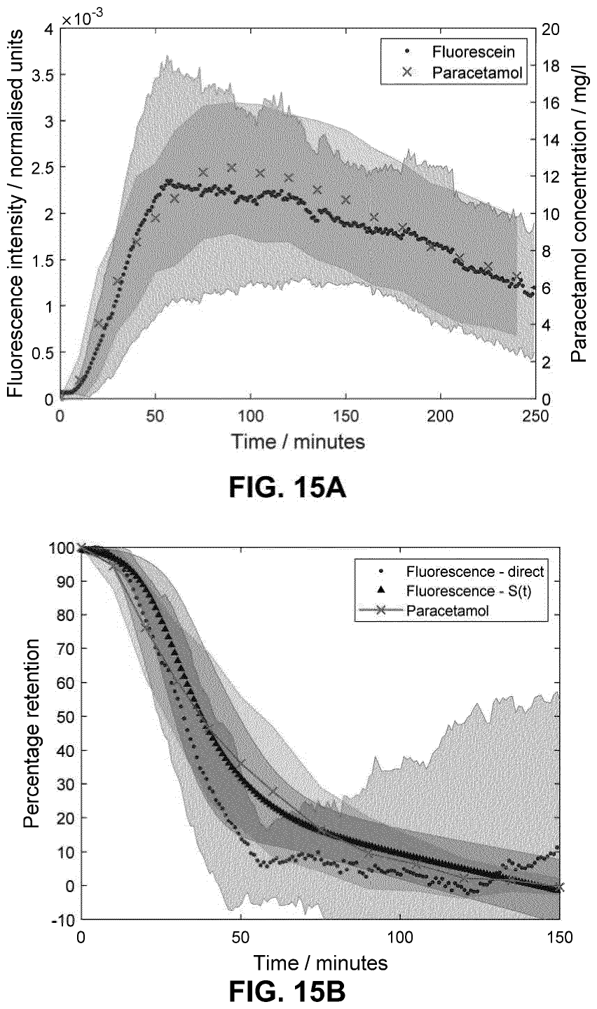

FIGS. 15A and 15B show a comparison of gastric emptying rate data obtained using a fluorescein-based measurement method as described above, and gastric emptying rate data obtained using a clinically approved paracetamol absorption test, performed in 20 healthy volunteers. The participants each consumed a liquid test meal (a milkshake) containing 500 mg fluorescein and 1.5 g paracetamol. Measurements of the transcutaneous fluorescence intensity were then made at one minute intervals. Blood samples were collected at 10-15 minute intervals for assessment of the serum paracetamol concentration.

FIG. 15A shows the fluorescence intensity and mean paracetamol concentration as functions of time. The shaded regions indicate one standard deviation from the mean. As shown, the fluorescence and paracetamol data exhibit a clear overlap for the full duration (250 minutes) of the experiments.

Comparing FIGS. 15A and 15B shows that the fluorescence versus time data (see FIG. 15A) can be converted to the percentage of the test meal which is remaining in the stomach of the subject as a function of time, R_{pc} (see FIG. 15B), by using the aforementioned calculation and fitting method for gastric emptying rate.

FIG. 15B shows the mean percentage retention (R_{pc}) values as functions of time for the paracetamol data (calculated according to the method of Medhus et. al as detailed in “Delay of gastric emptying by duodenal intubation: sensitive measurement of gastric emptying by the paracetamol absorption test”, first published 24 Dec. 2001: https://doi.org/10.1046/j.1365-2036.1999.00519.x) and the fluorescence data (calculated according to the methods described above). The shaded regions demarcate one standard deviation from the mean. The “Fluorescence−direct” data (represented by dots) represent the percentage retention calculated directly from the fluorescence intensity data (i.e. R_{pc_direct}, as described above). The “Fluorescence−S(t)” data (represented by triangles) represent the percentage retention calculated based on the fitting procedure described above with R_{pc} calculated according to the data in the variable S(t) (as detailed above).

As illustrated in FIGS. 15A and 15B, the above analysis can thus advantageously provide graphs and measurements that are similar to those used in clinical diagnosis of delayed gastric emptying (for example, gastric scintigraphy or paracetamol absorption tests), where the percentage of gastric contents retained in the stomach is plotted as a function of time and used for diagnostic assessment. Advantageously, using the method described herein, this can be achieved using far less invasive procedures.

Various modifications may be made to the described embodiment(s) without departing from the scope of the invention as defined by the accompanying claims.

1. A method for measuring gut permeability of a subject, the method comprising:
orally administering a solution comprising a fluorescent contrast agent that is absorbable by a healthy gut to the subject;
using a light source to irradiate a location on the skin on a body part of the subject using light radiation, such that the light radiation causes at least a portion of the solution which has leaked out of the gut of the subject into the bloodstream of the subject to fluoresce;
using a transcutaneous sensing device to periodically detect the intensity of the fluorescence of the solution at said location to obtain fluorescence data of said intensity as a function of time;
normalising the fluorescence data to obtain normalised data of said intensity as a function of time; and
analysing said normalised data to determine the gut permeability of the subject by calculating one or more of:
a) the first peak value of said intensity;
b) the integral of said intensity with respect to time;
c) the product of the first peak value of said intensity and the time at said peak value;
d) the product of: the first peak value of said intensity, and the time at a point past the time of said first peak value;
e) the time at which the first peak value of said intensity occurs; or
f) the first peak value of said intensity divided by the time at which said peak value occurs.

2. A method as claimed in claim 1, wherein the solution comprises water or juice.

3. A method as claimed in claim 1, wherein the contrast agent comprises a dye, for example fluorescein, methylene blue or fluorescein isothiocyanate conjugated dextran, or salts thereof, or combinations thereof.

4. A method as claimed in claim 1, wherein the transcutaneous sensing device has an acquisition time and the light source has an excitation power, and wherein the fluorescence data is normalised based on said acquisition time and said excitation power.

5. A method as claimed in claim 1, wherein the transcutaneous sensing device begins taking periodic measurements before the solution is administered to the subject, such that the periodic detection of the intensity of the fluorescence of the solution begins before the solution is administered to the subject, to obtain a background signal to be used in the step of normalising the fluorescence data.

6. A method as claimed in claim 1, wherein the light source comprises a light emitting diode or a laser.

7. A method as claimed in claim 1, wherein the transcutaneous sensing device comprises one or more photodiodes, phototransistors and/or fibre-optic probes and is configured to be worn on and/or around said body part of the subject.

8. A method as claimed in claim 1, wherein in the step of using the transcutaneous sensing device to periodically detect said intensity of the fluorescence, measurements are recorded by said transcutaneous sensing device at least once per minute.

9. A method as claimed in claim 1, wherein said body part is a finger, wrist, arm or earlobe.

10. A method for measuring gastric emptying rate of a subject, the method comprising:
orally administering a test meal comprising a fluorescent contrast agent that is absorbable by a healthy gut to the subject;
using a light source to irradiate a location on the skin on a body part of the subject using light radiation, such that the radiation causes at least a portion of the fluorescent contrast agent in the test meal which has been emptied from the stomach of the subject and has entered the bloodstream of the subject to fluoresce;
using a transcutaneous sensing device to periodically detect the intensity of the fluorescence of the fluorescent contrast agent in the test meal at said location to obtain data of said intensity as a function of time;
normalising the fluorescence data to obtain normalised data of said intensity as a function of time; and
analysing said normalised data to calculate the percentage of the test meal which is remaining in the stomach of the subject as a function of time, based on the intensity as a function of time divided by a peak value of the intensity.

11. A method as claimed in claim 10, wherein the peak value of the intensity is the value of the intensity at a first peak or at a second peak in the fluorescence data, or is a maximum value of the intensity in the fluorescence data.

12. A method as claimed in claim 10, wherein the test meal comprises a liquid test meal or a solid test meal.

13. A method as claimed in claim 10, wherein the contrast agent comprises a dye, for example fluorescein, methylene blue or fluorescein isothiocyanate conjugated dextran, or salts thereof, or combinations thereof.

14. A method as claimed in claim 10, wherein the transcutaneous sensing device has an acquisition time and the light source has an excitation power, and wherein the fluorescence data is normalised based on said acquisition time and said excitation power.

15. A method as claimed in claim 10, wherein the transcutaneous sensing device begins taking periodic measurements before the test meal is administered to the subject, such that the periodic detection of the intensity of the fluorescence of the test meal begins before the test meal is administered to the subject, to obtain a background signal to be used in the step of normalising the fluorescence data.

16. A method as claimed in claim 10, wherein the light source comprises a light emitting diode or a laser.

17. A method as claimed in claim 10, wherein the transcutaneous sensing device comprises one or more photodiodes, phototransistors and/or fibre-optic probes and is configured to be worn on and/or around said body part of the subject.

18. A method as claimed in claim 10, wherein in the step of using the transcutaneous sensing device to periodically detect said intensity of the fluorescence, measurements are recorded by said transcutaneous sensing device at least once per minute.

19. A method as claimed in claim 10, wherein said body part is a finger, wrist, arm or earlobe.

20. A method as claimed in claim 10, wherein the step of analysing said normalised data comprises fitting the function:
$$I ( t ) = ( 1 - Ct ) ( \frac{{B}_{max}}{1 + exp ( - {k}_{B} ( t - {t}_{B \frac{1}{2}} ) )} + \frac{{L}_{max}}{1 + exp ( - {k}_{L} ( t - {t}_{L \frac{1}{2}} ) )} )$$
onto the normalised data using a numerical fitting procedure such as least squares fitting;
wherein:
t represents time;
I(t) represents the normalised data of said fluorescence intensity as a function of time;
Bₘₐₓ represents the maximum value of the intensity contribution from the fluorescent contrast agent in the bloodstream of the subject as a function of time;
Lₘₐₓ represents the maximum value of the intensity contribution from the fluorescent contrast agent that has leaked into the epithelium of the skin of the subject as a function of time;
t_{B1/2} represents the time at which the intensity contribution from the fluorescent contrast agent in the bloodstream of the subject as a function of time reaches half of its maximum value;
t_{L1/2} represents the time at which the intensity contribution from the fluorescent contrast agent that has leaked into the epithelium of the skin of the subject as a function of time reaches half of its maximum value;
C represents the rate at which dye is eliminated from the body of the subject;
k_{B} is a constant and represents the logistic growth rate of the intensity contribution from the fluorescent contrast agent in the bloodstream of the subject as a function of time; and
k_{L} is a constant and represents the logistic growth rate of the intensity contribution from the fluorescent contrast agent that has leaked into the epithelium of the skin of the subject as a function of time.

21. A method as claimed in claim 20, wherein the step of analysing said normalised data further comprises calculating the amount of dye that has emptied from the stomach of the subject as a function of time, S(t), wherein S(t)=B(t)+B(t)Ct, wherein B(t) represents the intensity contribution from the fluorescent contrast agent in the bloodstream of the subject as a function of time.

22. A method as claimed in claim 21, wherein the step of analysing said normalised data further comprises calculating the percentage of the test meal which is remaining in the stomach of the subject as a function of time, R_{pc}, wherein:
$${R}_{p c} = 100 ⨯ ( 1 - \frac{S ( t )}{S ( {t}_{peak} )} )$$
and wherein S(tₚₑₐₖ) represents the value of S(t) at the time at which a final peak is observed in the normalised data of said intensity as a function of time.

23. A method as claimed in claim 20, wherein the step of analysing said normalised data further comprises calculating the percentage of the test meal which is remaining in the stomach of the subject as a function of time, R_{pc}, wherein:
$${R}_{p c} = 100 ⨯ ( 1 - \frac{B ( t )}{{B}_{max}} )$$
and wherein B(t) represents the intensity contribution from the fluorescent contrast agent in the bloodstream of the subject as a function of time.

24. A method as claimed in claim 10, wherein the step of analysing said normalised data comprises calculating the percentage of the test meal which is remaining in the stomach of the subject as a function of time, R_{pc}, wherein:
$${R}_{p c} = 100 ⨯ ( 1 - \frac{I ( t )}{I ( {t}_{peak 1} )} )$$
and wherein I(t) represents the normalised data of said intensity as a function of time; and
tₚₑₐₖ₁ represents the time at which the first peak is observed in said intensity as a function of time.
